(19) United States
(12) Patent Application Publication
(10) Pub. No.: US 2005/0238658 A1
(43) Pub. Date: Oct. 27, 2005

(54) Transposon

(76) Inventors: Duncan John Maskell, Cambridge (GB); Ian George Charles, London (GB); Andy Allen, London (GB); Paul Owen, London (GB)

Correspondence Address: NIXON & VANDERHYE, PC, 901 NORTH GLEBE ROAD, 11TH FLOOR, ARLINGTON, VA, 22203, US

(21) Appl. No.: 10/506,794

(22) PCT Filed: Mar. 5, 2003
(86) PCT No.: PCT/GB03/00918

§ 371 Date: Apr. 15, 2005

(30) Foreign Application Priority Data
Mar. 5, 2002 (GB) 0205143.1
Sep. 26, 2002 (GB) 0222378.2

(51) Int. Cl. A61K 39/02; C12Q 1/68; C12N 15/74; C12N 1/21

(52) U.S. Cl. 424/200.1; 435/473; 435/6; 435/252.3

(57) ABSTRACT

A transposon which comprises an RNA polymerase recognition site and a homing endonuclease recognition site. The transposon may be used in methods for the identification of an essential gene or a conditional essential gene of an organism. Genes identified in such methods are useful as substrates for use in screening for antibacterials, antiparasitics, fungicides, pesticides and herbicides.

### FIELD OF THE INVENTION

This invention relates to a new type of transposon. The transposon may be used in methods for the identification of essential and conditional essential genes, in particular in bacteria.

### BACKGROUND OF THE INVENTION

The increase in prevalence of antibiotic-resistant bacteria, for example, has renewed interest in the search for new targets for antibacterial agents. Essential genes and in particular the proteins which they encode may be good substrates for use in screens for antibacterials, antiparasitics, fungicides, pesticides and herbicides. Essential genes and their protein products potentially represent such targets.

Additionally, there is an interest in the identification of conditional essential genes, that is genes which are essential for the survival of an organism in a particular environment. In the case of pathogenic bacteria, for example, conditional essential genes include those which are required for survival in a host. Such genes and the proteins which they encode may also be good targets for use in screens for antibacterials. In particular, bacteria which carry mutations in such genes may be useful in attenuated live vaccines.

### SUMMARY OF THE INVENTION

Essential genes are those genes which, when missing (eg. because of a chromosomal deletion) or mutated to render them non-functional, result in a lethal phenotype. That is, they are genes without which an organism cannot survive. Conditional essential genes are those genes which, although not absolutely essential for the survival of an organism under all conditions, are essential for survival under various conditional restraints. Examples of particular conditional restraints include survival at elevated temperatures and survival of a pathogen within its host.

A number of transposon-based strategies have been developed for the identification of essential and conditional essential genes. We have now devised a new set of transposons. The transposons can be used in a variety of methods for the identification of essential and conditional essential gene in the genome of an organism.

According to the invention there is thus provided a transposon which comprises an RNA polymerase recognition site and a homing endonuclease recognition site.

The invention also provides:

use of a transposon of the invention in a method for the identification of an essential or a conditional essential gene;

a method for identifying an essential gene of an organism, which method comprises:

(i) providing a library of transposon insertion mutants of the said organism, wherein the transposon is a transposon of the invention;

(ii) isolating chromosomal DNA from the library of (i);

(iii) digesting the chromosomal DNA with a restriction endonuclease that is capable of cutting 5′ to the RNA polymerase recognition site(s) in the transposon and 3′ to the RNA polymerase recognition site(s) in the chromosomal DNA flanking the transposon insertion site;

(iv) transcribing the resulting digested DNA from the RNA polymerase recognition site(s) in the said DNA;

(v) hybridising the resulting RNA with an oligonucleotide array; and

(vi) identifying a probe on the oligonucleotide array which corresponds to an essential gene of the organism;

a method for identifying a conditional essential gene of an organism, which method comprises:

(a) providing a first sample of a library of transposon insertion mutants of the said organism (input library);

(b) providing a second sample of the library and subjecting that sample to a conditional restraint;

(c) collecting the mutants that survive the conditional restraint in step (ii) to give a second library (output library);

(d) carrying out a method according to steps (ii) to (iv) of the method set out above on the input library from step (a) and on the output library from step (c);

(e) hybridising the transcribed RNA derived from the input library and from the output library separately to copies of the same oligonucleotide array or, if the RNA derived from the two libraries is differentially labelled, to the same oligonucleotide array; and

(f) identifying a probe on the oligonucleotide array(s) which corresponds to a conditional essential gene of the organism;

a method for identifying an essential gene of an organism, which method comprises:

(i) providing a library of transposon insertion mutants of the said organism, wherein the transposon is a transposon of the invention;

(ii) isolating chromosomal DNA from the library of (i);

(iii) digesting the chromosomal DNA with a restriction endonuclease that is capable of cutting 5′ to the RNA polymerase recognition site(s) in the transposon and 3′ to the RNA polymerase recognition site(s) in the chromosomal DNA flanking the transposon insertion site;

(iv) transcribing the resulting digested DNA from the RNA polymerase recognition site(s) in the said DNA;

(v)′ reverse transcribing the resulting RNA;

(v)″ hybridising the resulting cDNA with an oligonucleotide array; and

(vi) identifying a probe on the oligonucleotide array which corresponds to an essential gene of the organism;

a method for identifying a conditional essential gene of an organism, which method comprises:

(a) providing a first sample of a library of transposon insertion mutants of the said organism (input library);

(b) providing a second sample of the library and subjecting that sample to a conditional restraint;

(c) collecting the mutants that survive the conditional restraint in step (ii) to give a second library (output library);

(d) carrying out a method according to steps (ii) to (v)′ of the method set out above on the input library from step (a) and on the output library from step (c);

(e) hybridising the reverse transcribed cDNA derived from the input library and from the output library separately to copies of the same oligonucleotide array or, if the cDNA derived from the two libraries is differentially labelled, to the same oligonucleotide array; and

(f) identifying a probe on the oligonucleotide array(s) which corresponds to a conditional essential gene of the organism;

use of an essential or conditional essential gene identified by a method as set out above, or a polypeptide encoded by a said gene, in a method for identifying an inhibitor of transcription and/or translation of that gene and/or activity of a polypeptide encoded that gene;

a method for identifying an inhibitor of transcription and/or translation of an essential or conditional essential gene and/or an inhibitor of activity of a polypeptide encoded by a said gene, which method comprises:

(a) identifying an essential or conditional essential gene by a method as set out above; and

(b) determining whether a test substance can inhibit transcription and/or translation of a gene identified in step (a) and/or activity of a polypeptide encoded by a said identified gene, thereby to identify a said inhibitor;

an inhibitor identified by such a method according to claim;

an inhibitor of the invention for use in a method of treatment of a bacterial, fungal or eukaryotic parasite infection, wherein the essential or conditional essential gene used to identify the inhibitor is a bacterial, fungal or eukaryotic parasite essential or conditional essential gene;

use of such an inhibitor in the manufacture of a medicament for use in the treatment of a bacterial, fungal or eukaryotic parasite infection;

a pharmaceutical composition comprising such an inhibitor and a pharmaceutically acceptable carrier or diluent;

a method of treating a host suffering from a bacterial, fungal or eukaryotic parasite infection, which method comprises the step of administering to the host a therapeutically effective amount of such an inhibitor;

a method for the preparation of a pharmaceutical composition, which method comprises:

(a) identifying an inhibitor of transcription and/or translation of an essential or conditional essential gene of an organism and/or an inhibitor of activity of a polypeptide encoded by a said gene by a method as set out above wherein the essential or conditional essential gene is a bacterial, fungal or eukaryotic parasite essential or conditional essential gene; and

(b) formulating the inhibitor thus identified with a pharmaceutically acceptable carrier or diluent;

a method for treating a host suffering from a bacterial, fungal or eukaryotic parasite infection, which method comprises:

(a) identifying an inhibitor of transcription and/or translation of an essential or conditional essential gene of an organism and/or an inhibitor of activity of a polypeptide encoded by a said gene by a method as set out above wherein the essential or conditional essential gene is a bacterial, fungal or eukaryotic parasite essential or conditional essential gene;

(b) formulating the inhibitor thus identified with a pharmaceutically acceptable carrier or diluent; and

(c) administering to the host a therapeutically effective amount of an inhibitor thus formulated;

an inhibitor of the invention, wherein the essential or conditional essential gene is a plant bacterial, plant fungal or plant pest essential or conditional essential gene;

use of such an inhibitor as a plant bactericide, fungicide or pesticide;

an inhibitor of the invention, wherein essential or conditional essential gene is a plant essential or conditional essential gene;

use of such an inhibitor as a herbicide;

a bacterium attenuated by a non-reverting mutation in one or more genes identified by a method of the invention;

a vaccine comprising such a bacterium and a pharmaceutically acceptable carrier or diluent;

a bacterium as described above for use in a method of vaccinating a human or animal;

use of such a bacterium for the manufacture of a medicament for vaccinating a human or animal;

a method for raising an immune response in a mammalian host, which method comprises the step of administering to the host a bacterium as set out above;

a method for preparing an attenuated bacterium, which method comprises:

(a) identifying a conditional essential gene in a bacterium by a method of the invention; and

(b) introducing a non-reverting mutation into a thus-identified conditional essential gene of the bacterium, thereby to attenuate the bacterium;

a method for the preparation of a vaccine, which method comprises:

(a) identifying a conditional essential gene in a bacterium by a method of the invention;

(b) introducing a non-reverting mutation into a thus-identified conditional essential gene of the bacterium, thereby to attenuate the bacterium; and

(c) formulating the attenuated bacterium with a pharmaceutically acceptable carrier or diluent; and

a method for raising an immune response in a mammalian host, which method comprises:

(a) identifying a conditional essential gene in a bacterium by a method of the invention;

(b) introducing a non-reverting mutation into a thus-identified conditional essential gene of the bacterium, thereby to attenuate the bacterium;

(c) formulating the attenuated bacterium with a pharmaceutically acceptable carrier or diluent; and

(d) administering to the host the attenuated bacterium thus formulated.

### BRIEF DESCRIPTION OF THE FIGURES

FIG. 1 shows a diagrammatic representation of the “Gene Kelly” transposon consisting of ME (mosaic end IS sequences), binding sites for the SP6 and T7 RNA polymerases, homing endonuclease sites for I-SceI and PI-PspI, an R6k origin of replication and a kanamycin resistance cassette.

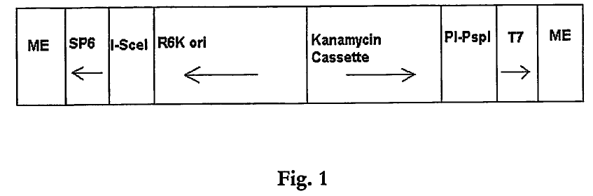

FIG. 2 sets out the sequence of the “Gene Kelly” transposon.

FIG. 3 shows a diagrammatic representation of the original epicentre EZ:Tn R6k ori Kan transposon with the oligonucleotide sequences overlaid. Black boxes represent matching sequence between PCR oligonucleotides and the epicentre transposon.

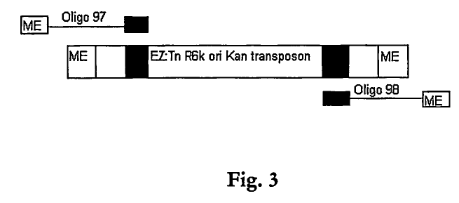

FIG. 4 sets out a graph showing the position of insertion in the LT2 genome of the 46 sequenced transposon mutants (position in base pairs in increasing number order against number of transposons). From the graph it can be seen that there is a fairly random distribution of insertions throughout the genome.

FIG. 5 sets out a diagram of each end of the transposon showing the relative position of the iPCR oligonucleotides with the restriction endonuclease cut sites and RNA polymerase promoters in transposed chromosomal DNA, which has been digested and religated.

FIG. 6 sets out a schematic diagram of the ligation capture method of recovering the ends of the transposon.

FIG. 7 sets out array hybridisation results comparing input and output pool data. Array Images, panel a, reveals comparable sections of the scanned microarrays resulting from the hybridisation of the labeled RNA target generated from the input and output pool restricted DNA. Sets of probes corresponding to three transposon mutants (a set of probes refers to probes synthesised in both the sense and anti-sense directions around the point of transposon insertion. These are shown as horizontal lines above, sense probes, and below, anti-sense probes, the disrupted locus), 1 (corresponding to probes encompassing a transposon insertion within the aroA gene), 2 (corresponding to probes encompassing a transposon insertion within gene X) and 3 (corresponding to probes encompassing a transposon insertion within an intergenic region) are boxed in the input panel and the data extracted from the input and output arrays are compared in the adjacent panel b, Extracted Data. In panel b, dashed vertical lines shown between the sense and anti-sense probes refer to the position of RsaI restriction endonuclease recognition sequences. Boxes above and below the black line indicate genes either in the sense or anti-sense direction, respectively, relative to the published LT2 genome sequence.

FIG. 8 sets out insertion site relative to S. aureus MW2 genome sequence of 59 Tn917, 50 Tn551 and 86 Mariner Erm Gene Kelly mutants generated in S. aureus SH1000.

### DESCRIPTION OF THE SEQUENCE LISTING

SEQ ID NO: 1 sets out the sequence of the “Gene Kelly” transposon.

SEQ ID NO: 2 sets out the sequence of primer 97, which was used in the construction of the “Gene Kelly” transposon.

SEQ ID NO: 3 sets out the sequence of primer 98, which was used in the construction of the “Gene Kelly” transposon.

SEQ ID NO: 4 sets out the sequence of primer 107, which can be used to carry out iPCR in a protocol for generating RNA run-offs.

SEQ ID NO: 5 sets out the sequence of primer 115, which can be used to carry out iPCR in a protocol for generating RNA run-offs.

SEQ ID NO: 6 sets out the sequence of primer 116, which can be used to carry out iPCR in a protocol for generating RNA run-offs.

SEQ ID NO: 7 sets out the sequence of primer 108, which can be used to carry out iPCR in a protocol for generating RNA run-offs.

SEQ ID NO: 8 sets out the sequence of primer 117, which can be used to carry out iPCR in a protocol for generating RNA run-offs.

SEQ ID NO: 9 sets out the sequence of primer 118, which can be used to carry out iPCR in a protocol for generating RNA run-offs.

SEQ ID NO: 10 sets out the sequence of primer 113, which can be used in a protocol for ligation capture recovery of “Gene Kelly” transposon ends.

SEQ ID NO: 11 sets out the sequence of primer 114, which can be used in a protocol for ligation capture recovery of “Gene Kelly” transposon ends.

SEQ ID NO: 12 sets out the sequence of primer 135, which was used in the construction of the “Mariner Erm Gene Kelly” transposon.

SEQ ID NO: 13 sets out the sequence of primer 136, which was used in the construction of the “Mariner Erm Gene Kelly” transposon.

SEQ ID NO: 14 sets out the sequence of primer 5′ erm, which can be used in the isolation of an erythromycin resistance marker gene sequence.

SEQ ID NO: 15 sets out the sequence of primer 3′ erm, which can be used in the isolation of an erythromycin resistance marker gene sequence.

SEQ ID NO: 16 sets out the sequence of primer 12, which can be used to sequence the “Mariner Erm Gene Kelly” transposon.

SEQ ID NO: 17 sets out the sequence of primer 13, which can be used to sequence the “Mariner Erm Gene Kelly” transposon.

SEQ ID NO: 18 sets out the sequence of primer 199, which was used to sequence chromosomal DNA from S. aureus which flanked Mariner Erm Gene Kelly transposon insertion sites.

### DETAILED DESCRIPTION OF THE INVENTION

The invention relates to a new transposon which is suitable for use in methods for the identification of essential and conditional essential genes. The transposon has been named the “Gene Kelly” transposon.

The transposon of the invention is typically a modified Tn5 or Mariner transposon, although, in principle, any transposon may be modified so as to prepare a transposon according to the invention. The transposon of the invention has a combination of features which make it a versatile tool for use in a number of protocols for the identification of essential and conditional essential genes. We refer to these methods as transposon mediated differential hybridisation (TMDH) techniques.

A transposon of the invention comprises an RNA polymerase recognition site (sometimes referred to as an RNA polymerase recognition sequence) and a honing endonuclease recognition site (sometimes referred to as a homing endonuclease recognition sequence).

Typically, the RNA polymerase recognition site is located proximal to an end of the transposon, for example adjacent to mosaic ends (if the transposon has them). The RNA polymerase recognition site is typically oriented so that it directs transcription out of the transposon itself. Thus, DNA sequence flanking the integration site of the transposon may be transcribed.

These transcribed sequence originate from DNA sequence flanking a transposon insertion site. Such DNA sequences are therefore susceptible to insertion and are unlikely to represent essential gene sequences.

Sequences flanking a transposon insertion site may therefore be isolated from a library of transposon insertion mutants (generated using a transposon of the invention) and hybridised with an oligonucleotide array which comprises probes corresponding to open reading frames from an organism to be studied. If the library of insertion mutants comprises a transposon insertion in all of the non-essential genes of the organism, any probe in the oligonucleotide array to which none of the flanking sequences hybridise is likely to be a good candidate for originating from an essential gene. Typically, oligonucleotide arrays suitable for use with a transposon comprise probes corresponding to all of the open reading frames from the organism in question and therefore potentially all of the essential genes of an organism may be identified simultaneously.

Typically, a transposon of the invention comprises two RNA polymerase recognition sites located proximal to the ends, typically to the opposite ends, of the transposon. Preferably, both RNA polymerase recognition sites point out of the transposon, i.e. are capable of directing transcription or DNA sequence flanking the transposon insertion site.

Preferred transposons of the invention comprise two diverse (different) RNA polymerase recognition sites, although the two sites may be the same. The use of two RNA polymerase recognition sites allows two separate pools of RNAs to be isolated from a library of Gene Kelly insertion mutants. One pool will correspond to DNA sequences flanking one side of the transposon insertion site and the other pool will represent sequences flanking the other side of the transposon insertion site (these may be referred to as right- and left-hand pools). The generation of these two separate pools may help to minimise the risk of an essential gene being incorrectly assigned as a non-essential gene. This is explained in more detail below.

A transposon of the invention also comprises a homing endonuclease recognition site. Homing endonucleases are rare cutters, especially in bacterial DNA. Incorporation of recognition sites for such endonucleases into a transposon effectively permits the introduction of these sites into the genome being studied. Transposed DNA may be digested with the appropriate homing endonuclease and the resulting ends (if none are present in the bacterial genome) should therefore all originate from the Gene Kelly transposon. The fragments resulting from digestion with a homing endonuclease may then be digested with a restriction endonuclease which cuts in the genome of organism being studied. The resulting transposon:flanking sequence fragments may be rescued using a ligation-capture technique described below, allowing the rapid and selective purification of regions of the genomic DNA of the organism being studied which originate from a site of transposon insertion. Such regions can be used in hybridisation experiments with oligonucleotide arrays as outlined above and as described in more detail below.

Preferably, if the transposon comprises two RNA polymerase recognition sites, it will also comprise two homing endonuclease recognition sites. Typically, if two homing endonuclease recognition sites are present, they will be diverse (different) homing endonuclease recognition sites. However, they may be the same endonuclease recognition site. The use of two, typically diverse, homing endonuclease recognition sites in combination with two, typically diverse, RNA polymerase recognition sites may allow two separate pools of sequences flanking either side of the inserted transposons to be isolated separately i.e. a left-hand pool and a right-hand pool of flanking sequences may be separated. The generation of these two pools may help to minimise the risk of an essential gene being incorrectly assigned as a non-essential gene. This is explained in more detail below.

In addition, a transposon of the invention may incorporate a bacterial origin of replication. This allows plasmid-rescue of the complete transposon to be carried out (plus the flanking regions of chromosomal DNA around the insertion site). This may be achieved by digestion of genomic DNA isolated from a library of transposon insertion mutants (with a restriction endonuclease that does not cut in the transposon sequence or at least in the bacterial origin of replication in the transposon sequence), religation and then transformation into a strain of bacteria in which the origin of replication will function. A suitable bacterial origin of replication is the R6k origin of replication.

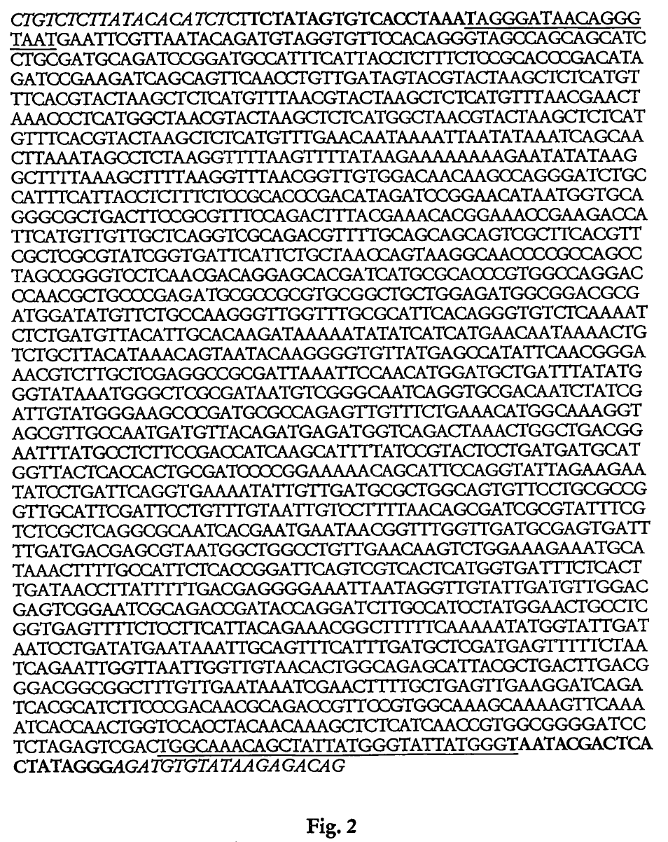

A transposon of the invention thus comprises two critical features: (i) an RNA polymerase recognition site; and (ii) a homing endonuclease recognition site. Optionally, a bacterial origin of replication may be present. A more preferred version of the Gene Kelly transposon comprises two diverse RNA recognition sites and two diverse homing endonuclease recognition sites. The full sequence of a preferred Gene Kelly transposon is set out in FIG. 2 and SEQ ID NO: 1.

Transposons, sometimes called transposable elements, are mobile polynucleotides. The term transposon is well known to those skilled in the art and includes classes of transposons that can be distinguished on the basis of sequence organisation, for example short inverted repeats at each end; directly repeated long terminal repeats (LTRs) at the ends; and polyA at 3′ ends of RNA transcripts with 5′ ends often truncated. Some types of virus also integrate into the host genome, for example retroviruses, and may therefore be used to generate libraries of insertion mutants. However, transposons are typically preferred to viruses because issues of safety related to pathogenicity may be avoided.

A transposon of the invention comprises an RNA polymerase recognition site (typically two) and a homing endonuclease recognition site (typically two). Any suitable transposon may be modified to produce a transposon of the invention. Suitable transposons for use in bacteria which can be modified to generate a Gene Kelly transposon include Tn3, γδ, Tn10, Tn5, TnphoA, Tn903, Tn917, Mariner Bacteriophage Mu and related viruses. Any of the above mentioned transposons may be modified to generate a transposon of the invention. Different parts of different transposons may be mixed and matched in a transposon of the invention. A particular preferred transposon for use in the invention is a modified Tn5 transposon.

Suitable transposons for use in fungi which can be modified to generate a transposon of the invention include the Ty1 element of Saccharomyces cerevisiae, the filamentous fungi elements (the filamentous fungi include agriculturally important plant pathogens such as Erysiphe graminis, Magnaporthe grisea) such as Fot1/Pogo-like and Tc1/Mariner-like elements (see Kempen and Kuck, 1998, Bioessays 20, 652-659 for a review of such elements).

Suitable transposons for use in plants which can be modified to generate a transposon of the invention include Ac/Ds, Tam3 and other Tam elements, cin4 and spm.

Suitable transposons for use in animals which can be modified to generate a transposon of the invention include P and hobo which may be used in Drosophila and Tc1 which can be used in Caenorhabditis elegans.

A preferred transposon of the invention is one which carries an antibiotic resistance gene (which may be useful in identifying mutants which carry a transposon) conferring resistance to, for example, kanamycin (in particular for use with a Tn5 -based transposon), erythromycin (in particular for use with a Mariner-based transposon) streptomycin or bleomycin.

A transposon of the invention may comprise one, two or more recognition sites for an RNA polymerase. Preferred recognition sites are those for which the corresponding RNA polymerase is highly selective for initiation. Other preferred recognition sites are those for which the corresponding RNA polymerase does not initiate transcription from sequences of the organism being studied. Preferred examples of RNA recognition sites are those recognised by bacteriophage RNA polymerases, for example the recognition site for T7 RNA polymerase, T3 RNA polymerase or SP6 RNA polymerase, in particular the T7 RNA polymerase recognition site. A preferred. Gene Kelly transposon will thus carry two of a 17 RNA polymerase recognition site, an SP6 RNA polymerase recognition site or a T3 RNA polymerase recognition site, for example a T7 RNA polymerase recognition site and an SP6 RNA polymerase recognition site. The recognition sites for these specific RNA polymerases are well known to those skilled in the art

The RNA polymerase recognition site may appear anywhere within a transposon for use in the invention. However, typically the RNA polymerase recognition site will be located proximal to one end of the transposon, i.e. proximal to one IS/ME. Typically, the 3′ end of the RNA polymerase recognition site will be situated from one to 30, for example from five to twenty base pairs away from the 5′ end of one of the IS/ME.

Preferred transposons of the invention comprise two RNA polymerase recognition sites, which generally will be different RNA polymerase recognition sites. For example, a transposon may comprise a T7 RNA polymerase recognition site and an SP6 RNA polymerase recognition site.

A transposon of the invention may also comprise more than two RNA polymerase recognition sites, for example three or four RNA recognition sites. More than two recognition sites may be useful in a situation where the genome of an organism being studied possesses recognition sites for one of the RNA polymerase recognition sites present in the transposon. Thus, different RNA polymerase sites in one transposon may be suitable for use in different genomes. The specific RNA polymerase recognition sites used may vary with the particular organism being studied. A single transposon may therefore be suitable for use in a number of different organisms.

A transposon of the invention comprises a homing endonuclease recognition site. Preferably, two such sites are present which may be the same or diverse. Homing endonucleases are also known as intron or intein encoded endonuclease. They are encoded by genes with mobile, self-splicing introns or inteins (protein introns). Any homing nuclease recognition site may be used, for example I-SceI, PI-PspI or I-PpoI, or preferably a combination of any two thereof.

A homing endonuclease recognition site may be located anywhere in the transposon, but typically it is situated 5′ to an RNA polymerase recognition site, i.e. further into the transposon than the RNA polymerase recognition site. Preferred transposons of the invention may comprise two homing endonuclease recognition sites which generally will be different homing endonuclease recognition sites, for example a I-SceI and a PI-PspI recognition site. This is usually the case where the transposon comprises two RNA polymerase recognition sites.

A transposon of the invention may, however, comprise more than two homing endonuclease recognition sites, for example three or four homing endonuclease recognition sites. More than two recognition sites may be useful in a situation where the genome of an organism being studied possesses recognition sites for one of the homing endonuclease recognition sites present in the transposon. Thus, different homing endonuclease recognition sites in one transposon may be suitable for use in different genomes. The specific homing endonuclease recognition sites used in the transposon may vary with the particular organism being studied. A single transposon may therefore be suitable for use in a number of different organisms.

Typically, a transposon of the invention will comprise the same number of RNA polymerase recognition sites as homing endonuclease recognition sites, ideally two of each. Each homing endonuclease recognition site will typically be located 5′ to an RNA polymerase recognition site, for example such that the 3′ end of the homing endonuclease recognition site is from one to 30, for example, from five to twenty base pairs away from the 5′ end of one of an RNA polymerase recognition site.

A transposon of the invention may also comprise a bacterial origin of replication. A preferred example of a suitable bacterial origin of replication is the R6k origin of replication. The R6k origin of replication is capable of functioning in a pir⁺ strain of bacteria.

A transposon of the invention may be used in methods for the identification of essential or conditional essential genes, i.e. use of a transposon of the invention in a method for the identification of an essential gene or a conditional essential gene is provided according to the invention. The methods described below for the identification of essential or conditional essential genes using a transposon of the invention can conveniently be referred to as transposon mediated differential hybridisation (TMDH). TMDH techniques are described in detail in WO-A-01/07651 (PCT/GB00/02879) and the transposon of the invention may be used in any of the methods set out therein.

In methods for the identification of essential gene, typically the first step is the provision of a library of transposon insertion mutants. Libraries of insertion mutants using a transposon of the invention may be generated according to any method known to those skilled in the art. For example, libraries of bacterial transposon insertion mutants can be constructed using either plasmid or bacteriophage vectors containing the transposon and a selectable marker. Bacteriophage λ, eg. λTnphoA can be used to infect a suitable recipient bacterial strain, for example E. coli XAC. This E. coli strain has a suppressor mutation which prevents the bacteriophage from replicating and subsequently lysing and also contains an antibiotic resistance gene to allow selection of colonies containing transposed chromosomal DNA. The vector contains mutation(s) preventing integration of the λ chromosome into the bacterial host chromosome and thus the growth of false positive colonies without a mutated E. coli gene is prevented. Cultures of the recipient strain are grown in enriched medium (eg. Luria Broth) and cells in mid log phase of growth are infected with the λ transposon vector for 1 hour at 37° C. Aliquots of the infected cells are plated out on L-agar supplemented with the appropriate selective antibiotic and grown overnight at 37° C. These colonies constitute a transposon library and can be further analysed by the TMDH procedure described in this application.

Alternatively, transposome complexes comprising the transposon in a complex with a transposase may be generated and electroporated into a suitable electrocompetent host. Suitable techniques for preparing transposomes and for electroporating transposomes into host cells are well known to those skilled in the art.

Growth of such libraries results in the generation of potentially thousands of insertion mutants all of which mutants carry insertion that are, of necessity, in genes that (when mutated) do not result in the death of the cell ie. are non-essential genes.

Each mutant in a suitable transposon insertion library may carry one transposon insertion. However, a mutant may carry more than one transposon insertion, for example two, three, four, five, ten or twenty transposon insertions. A transposon insertion mutant library suitable for use in the invention will comprise at least one transposon insertion mutant for at least 60%, at least 70%, typically at least 80%, preferably at least 90%, more preferably at least 95%, even more preferably at least 99%, or most preferably substantially all of the non-essential genes in the organism being studied. Preferably the library will be a saturating library, i.e. the library comprises a transposon insertion mutant for substantially all genes of the organism that when mutated give rise to viable organisms.

A transposon insertion could be in an open reading frame of a gene or in a regulatory sequence of gene.

Any non-essential gene in the transposon insertion library may be represented by more than one insertion mutant, for example two, three, four, five or up to ten insertion mutants, each carrying transposon insertions at the same or different sites in the non-essential gene or carrying insertions at the same site in different orientations. Preferred libraries will have, on average, more than one different transposon insertion mutant for each non-essential gene represented in the library, for example at least two on average, at least four on average, at least 5 on average or at least 10 on average different transposon insertion mutants for each non-essential gene represented in the library.

Some regions of a particular genome may be inaccessible to insertion by a particular transposon, for example because of a particular secondary or tertiary structure which is inaccessible to a particular transposon. Thus it may be advantageous to combine two transposon libraries, thereby increasing the probability of obtaining transposon insertions in a greater number of genes. For example, in the case of bacterial libraries, a library generated with Gene Kelly transposons based on a Tn5 transposon and a library generated with a Gene Kelly transposons based on a Tn10 transposon could, for example, be combined.

Generally, flanking sequence will be isolated from at least 60%, for example at least 70%, at least 80%, preferably at least 90%, more preferably at least 95% and most preferably at least 99% of the transposon insertion mutants in a particular library of mutants.

In the method of the invention chromosomal DNA is prepared from the library of transposon insertion mutants. Techniques for the isolation of chromosomal DNA, alternatively referred to as genomic DNA, are well known to those skilled in the art. The transposons of the invention allow for a number of different techniques to be used for the generation of RNA target sequences from the isolated genomic DNA.

In one version of TMDH, the chromosomal DNA thus prepared is then digested with a restriction endonuclease. The restriction endonuclease is one which is capable of cutting at a recognition site which is located in the transposon at a position 5′ to the RNA polymerase recognition site (which is located in the transposon) and 3′ to the RNA polymerase recognition site in the chromosomal DNA flanking the transposon insertion site.

In a modification of this protocol, two restriction endonucleases may be used: a first restriction enzyme which cuts 5′ to the RNA polymerase recognition site (which is located in the transposon) in the transposon itself; and a second restriction enzyme which cuts 3′ to the RNA polymerase recognition site in the chromosomal DNA flanking the transposon insertion site. If two restriction enzymes are used in this way, they may be used simultaneously or one after the other in either order.

The exact restriction enzyme(s) to be used will depend on the sequence of the transposon. However, typically an restriction endonuclease is used which has recognition sites that appear frequently within the genome of the organism being studied. Thus, a series of DNA fragments is generated, some of which comprise an RNA polymerase recognition site fused to a portion of flanking sequence, i.e. non-essential gene sequence.

Generally, suitable restriction endonucleases will have six base pair, five base pair or preferably four base pair recognition sequences. Suitable examples of four base pair cutters are set out in Table 1 below:

TABLE 1
Examples of 4 bp recognition type II restriction
endonucleases suitable for use in TMDH

| Enzyme | Recognition Site |
|---|---|
| AciI | C¹CGC |
| | GGC₁G |
| AluI | AG¹CT |
| | TC₁GA |
| BfaI | C¹TAG |
| | GAT₁C |
| BstuI | CG¹CG |
| | GC₁GC |
| DpnI | ¹GATC |
| | CTAG₁ |
| HaeIII | GG¹CC |
| | CC₁GG |
| HinpI | G¹CGC |
| | CGC₁G |
| HhaI | GCG¹C |
| | C₁GCG |
| MseI | T¹TAA |
| | AAT₁T |
| MspI | C¹CGG |
| | GGC₁C |
| NlaIII | ¹CATG |
| | GTAC₁ |
| RsaI | GT¹AC |
| | CA₁TG |
| Sau3a | ¹GATC |
| | CTAG₁ |
| TaqI | T¹CGA |
| | AGC₁T |
| Tsp509 | ¹AATT |
| | TTAA₁ |

The resulting fragments comprise an RNA polymerase site adjacent to non-essential gene sequence. The fragments may optionally be size selected. If size selection is carried out, fragments with a size of from about 100 bp to about 2000 bp or preferably of from about 200 bp to about 600 bp may be isolated, for example from a gel, and purified. The smaller the fragments isolated, the smaller the chance of the RNA target sequences including sequences from genes which lie next to genes which have been interrupted by transposons. If such adjacent sequences were from essential genes, there is the possibility that essential gene sequences could be identified as non-essential gene sequences. Thus, size fractionation may reduce the amount of false non-essential gene sequences.

RNA target sequences are generated from the DNA (host organism) sequences that flank the transposons, i.e. those regions corresponding to non-essential gene sequences. That is, the transposon:flanking sequence fragments are then used to generate the RNA sequences. Thus, following digestion, the transposon:flanking sequence fragments (the “target”) may be transcribed. Optionally, the transposon:flanking sequence fragments may be amplified prior to transcription, for example by PCR. Preferably this is carried out by iPCR (inverse PCR).

Transcription is carried out by in vitro transcription from the RNA polymerase recognition sequence. Techniques for carrying out in vitro transcription are well known to those skilled in the art and any suitable technique may be used. In essence, an RNA polymerase and ribonucleotides are used.

The RNA target sequences so-generated may then be hybridised with oligonucleotide arrays. Alternatively, the RNA target sequences may be reverse transcribed to produce cDNA target sequences. Reverse transcription may be primed using an oligonucleotide having a sequence based on the transposon sequence immediately 3′ to the site of transcription initiation. Techniques for carrying out reverse transcription are well known to those skilled in the art and any suitable technique may be used. In essence, a reverse transcriptase and deoxyribonucleotides are used.

Preferably, the transcription reaction or the reverse transcription reaction is carried out in the presence of one or more labelled ribonucleotides or one or more labelled deoxyribonucleotides respectively, so that the resulting RNA or cDNA target sequences are labelled.

Suitable labels include radioactive labels, for example ³²P, ³³P or ³⁵S, or non-radioactive labels, for example an enzyme, a fluorescent label or biotin.

Fluorescent labels are preferred, for example a water-soluble fluorescent dye such as Cy3™ or Cy5™ or a fluorescein-tagged compound such as FluorX™ (the NHS ester of carboxyfluorescein with an extended linker arm), fluorescein isothiocyanate (FITC) or 5-([4,6-Dichlorotriazin-2-yl]amino)fluorescein (DTAF). Generally, it will only be necessary to have one of the four ribonucleotides or deoxyribonucleotides labelled.

The techniques described above allow the isolation of sequences flanking the transposons in a library of transposon insertion mutants. Thus, a pool of flanking sequences is generated collectively referred to as the RNA (or cDNA) target sequences. Although fragments in the pool are generated from only one side of the transposons, a transposon is capable of inserting at any particular locus (that can be disrupted) in either orientation. Thus, particularly in a saturating transposon insertion library, many loci will be represented by mutants carrying insertions in both orientations. Therefore, the RNA (or cDNA) target sequences generated according to the TMDH method of the invention will, for many loci, comprise flanking sequence in both orientations.

In a modification of this technique, the fragments comprising the RNA polymerase site fused to non-essential gene sequence may be amplified.

Amplification may be carried out by ligating linkers, preferably vectorette units, to the fragments. If linkers are ligated to the fragments, the resulting fragments may be re-purified for example through a gel or by using spun-column chromatography. PCR may then carried out using the fragments as templates with a primer pair comprising an oligonucleotide specific for a transposon sequence and a second oligonucleotide specific for a linker (eg. a vectorette) sequence. The use of transposon- and vectorette-specific PCR primers results in the specific amplification of sequences that are adjacent to the sites of transposon insertion.

Alternatively, the fragments may be amplified by cycle primer extension. The use of a suitable labelled oligonucleotide primer can allow the amplification of sequences adjacent to the sites of transposon insertion. Those labelled amplified sequences can be used directly in hybridisation experiments.

Alternatively, the fragments may be amplified by inverse PCR (iPCR). Thus, the fragments may be self-ligated and subsequently amplified using transposon specific primers.

Suitable techniques for carrying out self-ligation are well known to those skilled in the art. Any suitable ligase may be used, for example T4 DNA ligase. Ligation reactions may be carried out for from 6 to 24 hours, for example from 12 to 16 hours at a temperature of from 10° C. to 20° C., for example at about 16° C.

Self-ligated molecules are then amplified using iPCR. Techniques for carrying out iPCR are well known to those skilled in the art and may be carried out according to any suitable technique. Typically, iPCR is carried out using two olignucleotides which bind divergently at a location 5′ to the RNA polymerase recognition site. Preferably the oligonucleotides bind divergently to a location which is 3′ to the restriction endonuclease recognition site in the transposon. That is, the two olignucleotide recognition sites are preferably located on the transposon between the restriction endonuclease recognition site and the RNA polymerase recognition site.

When using iPCR techniques, there is the possibility that, a “stuffer” fragment may ligate into the self-ligation reaction, which will be amplified along with the transposon-disrupted sequence. If this material were to be used in subsequent generation of the RNA target sequences, the stuffer sequence could create non-specific background signal as it would also be hybridized to the high density array. In order to remove this stuffer fragment, the sequences amplified in iPCR can be redigested with whichever enzyme was used to isolate the transposon-flanking sequence fragments in the first place. This results in the release of the stuffer fragments which can be removed from the transposon:flanking sequence fragments. Removal of the stuffer fragments can be facilitated if a biotinylated primer is used in iPCR The biotinylated transposon:flanking sequence fragments can then be removed from the stuffer fragments using a magnetic-bead-streptavidin conjugate.

Additional methods for amplifying transposon:flanking sequence fragments include, for example, splinkerette-PCR, targetted gene walking PCR, restriction site PCR, capture PCR, panhandle PCR and boomerang DNA amplification (for a review of these techniques see Hui et al., Cell Mol. Life Sci. 54 (1998) 1403-1411).

It is possible that the particular restricted endonuclease used will not cut within the gene in which the transposon is inserted, or cuts at a large distance, for example more than 2 kb, away from the insertion site. Therefore, if size selection is carried out, sequences from these genes may be lost. Thus, the generation of fragments may be carried out several times, each time using a different restriction endonuclease and the resulting fragments may subsequently be pooled. The greater the number of enzymes used to make fragments, the greater the likelihood of sequences from non-essential genes being represented in the final pool of fragments.

In a further modification of TMDH method, the chromosomal DNA isolated from a transposon insertion library may be divided into a number of aliquots. Those aliquots may then each be separately digested with a different restriction endonuclease which is capable of cutting at a recognition site which is located in the transposon at a position 5′ to the RNA polymerase recognition site and in the chromosomal DNA flanking the transposon 3′ to the RNA polymerase recognition site (which is in the transposon). The chromosomal DNA may be separated into, for example two, three, four, five or ten aliquots which are each separately digested with a different restriction endonuclease. Preferred restriction enzymes are as set out in Table 1 above.

Thus, for example, two or three aliquots of the chromosomal DNA may be separately digested with different suitable restriction endonucleases, for example two or three of HaeIII, HhaI, Hpych4IV and RsaI.

If the TMDH protocol is used in this modified format, the different aliquots may be repooled after digestion and treated together in the subsequent steps of TMDH. Alternatively, the digested aliquots may be treated separately in the subsequent steps. If this TMDH format is adopted, a number of pools of RNA target sequences result. Each pool of RNA target sequences may be labelled with a different, for example fluorescent, label.

In an alternative protocol for the preparation of RNA/cDNA target sequences, the genomic DNA isolated from a library of transposon mutants is digested with a homing endonuclease. Homing endonuclease recognition sites are rare and therefore any “ends” generated by digestion with a homing endonuclease should originate from the transposon. Fragments resulting from digestion with a homing endonuclease are then further digested with a restriction endonuclease which does not cut in the transposon, for example a restriction enzyme as described above, but which does cut within the genomic sequence flanking the transposon ends (i.e. flanking the transposon insertion site). The resulting transposon:flanking sequence fragments may then be rescued by annealing a biotinylated linker to them and then isolating the biotinylated transposon:flanking sequence fragments with streptavidin-coated particles, for example streptavidin-coated magnetic beads. The linker anneals to the homing endonuclease recognition site end of the fragments.

Alternatively the ends bearing the homing endonuclease recognition site may be rescued by labelling with digoxygenin and isolating the labelled fragments with an antibody raised against digoxygenin. The step of digestion with a restriction enzyme may be carried out after the transposon:flanking sequence fragments have been isolated.

The use of homing endonucleases may allow the step of isolating genomic DNA from a library of transposon mutants to be eliminated. Thus, the library of mutants may be digested directly with a homing endonuclease. Typically, an extract of the library may be generated. For example, if the library is a bacterial library, the baterial cells may be lysed before digestion with a homing endonuclease is carried out. Once digestion with a homing endonuclease has been carried out, transposon ends (transposon:flanking sequence fragments) may be recovered as described above.

The transposon:flanking sequence fragments are then used to generate the RNA target sequences by carrying out in vitro transcription from the RNA polymerase recognition site. In vitro transcription can be carried out as described above. If, after cutting with the homing endonuclease, a further restriction enzyme is not used to cut within the flanking sequences, in vitro transcription may be carried out in the presence of a dideoxyribonucleotide. That allows transcription to be terminated, reducing the risk of the transcribed sequence comprising portions of genes adjacent to the gene into which insertion has taken place.

The sequences which comprise the RNA target sequences (whichever of the methods described above is used to generate them) may be used for hybridisation with olignucleotide arrays. Oligonucleotide arrays used in the TMDH protocol of the invention are preferably high information content arrays.

Oligonucleotide arrays suitable for use in the invention may comprise sequences from one or more loci of a genome. Preferably suitable oligonucleotide arrays will represent at least 80% of all open reading frames (ORFs), more preferably at least 90% of all ORFs, for example 95% of all ORFs, even more preferably 99% of all ORFs or substantially all ORFs of the genome represented on the oligonucleotide array.

By high information content array is meant an array in which there are a high number of probes covering the locus, loci or genome represented by the array. For example, in a high information content array there may be a probe, for example, for every 30 to 500 base pairs of the locus, loci or genome represented by the array. Preferably there will be a probe, for every 60 to 250 base pairs of locus, loci or genome represented in the array, for example about every 100 base pairs. Probes may overlap, for example by 1, 2, 3, 4, 5, up to 10, up to 20, up to 30, up to 40 or up to 50 bases.

The olignucleotide probes on the array are, for example, from about 8 or 9 to about 150 nucleotides in length, preferably from about 30 or 50 to about 100 nucleotides in length or more preferably about 60 nucleotides in length.

The oligonucleotide probes used in the array will typically be designed on the basis of the wild type sequence of the organism being studied. The oligonucleotide probes may be designed so that each probe has minimal or substantially no cross-hybridisation with other sequences in the genome from which the probes originate. The BLAST program can be used to design suitable probes (Altschul et al., J. Mol. Biol. 215. 403-410).

Methods for making oligonucleotide arrays are well known to those skilled in the art.

Probes which show no hybridisation or substantially no hybridisation (there may be a low level of background non-specific hybridisation) with the RNA target represent sequences that are unlikely to have been disrupted by a transposon insertion event and consequently are strong candidates for sequences corresponding to essential genes.

However, it is theoretically possible for oligonucleotide probes within the 5′ or 3′-termini of essential genes to show a hybridisation signal with the TMDH protocol. For example, if a transposon insertion occurs in a non-essential gene adjacent to an essential gene, RNA target sequences may be generated from this transposon corresponding to both non-essential and essential gene sequences as a result of restriction sites lying within the essential gene. The resulting labelled target will not only comprise DNA corresponding to the non-essential gene (that has been disrupted), but will also extend into the adjacent essential gene up to the restriction site. The result of hybridising this labelled target to the oligonucleotide array will be appear as “bleed through” of signal to probes on either the 5′ or 3′ end of the essential gene, up to the restriction site used for the TMDH protocol.

To address this potential source of mis-assignment of essential genes, the restriction endonuclease digestion TMDH protocol described above may be carried with more than one aliquot of the isolated genomic DNA, for example two or three, whereby each aliquot is digested with a different restriction endonuclease (which have different recognition motifs). The more aliquots digested, with more restriction sites that are used to generate target sequences, the more statistically unlikely it is that all of them will result in labelled RNA target sequences that “bleed through” into essential genes. The pools of RNA target sequences derived from the different digestions can be hybridised to the same or, preferably, different oligonucleotide arrays if they were generated using different labels, or alternatively may be hybridised to copies of the same or, preferably, a different array. The analysis of the resulting multiple array hybridisation patterns will remove any ambiguity on the site of transposon insertion.

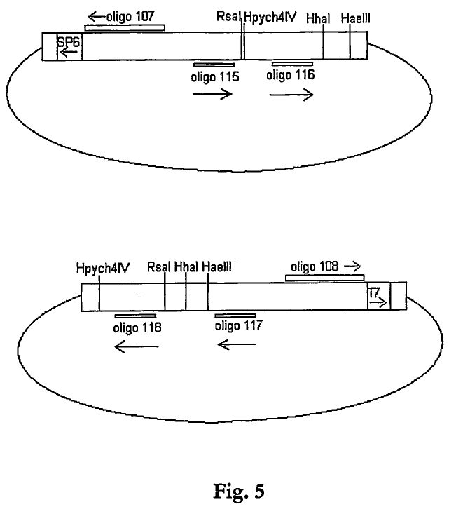

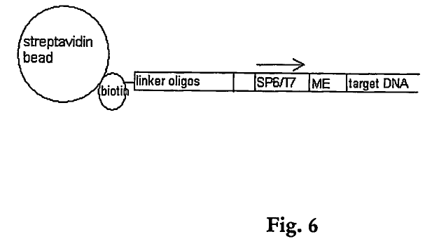

A different modification to the TMDH protocol, applicable to both the standard restriction endonuclease and the homing endonuclease approaches, to minimize the mis-assignment of essential genes as non-essential gene is to isolate two pools of RNA target sequences which each originate from different sides of the transposons. This approach is illustrated in FIGS. 5 and 6. FIGS. 5 and 6 show Gene Kelly transposons which comprise two RNA polymerase recognition sequences (although in FIG. 6 the two ends are shown superimposed).

In FIG. 5 one pool of RNA target sequences (from one side of the transposons) is generated by carrying out in vitro transcription using T7 polymerase and a second pool of RNA target sequences (from the other side of the transposon) is generated by carrying out in vitro transcription using T3 or SP6 polymerase.

Similarly, in FIG. 6 one pool of RNA target sequences (from one side of the transposons) is generated by carrying out in vitro transcription using T7 polymerase and a second pool of RNA target sequences (from the other side of the transposon) is generated by carrying out in vitro transcription using T3 or SP6 polymerase. The ends lying 5′ to the RNA polymerase recognition site (which are rescued using streptavidin coated particles) are generated via digestion with homing endonucleases. The homing endonuclease recognition site associated with each RNA polymerase recognition site may be the same or different homing endonuclease recognition sites.

The two pools of RNA target sequences can be hybridised to the same oligonucleotide array if they were generated using different labels, or alternatively each may be hybridised to a separate copy of the same array. Probes in the oligonucleotide array which shows no hybridisation to either pool are likely to correspond to essential genes.

Where an essential gene sequence is isolated in one of the RNA/cDNA pools because it lies close to a non-essential gene sequence flanking a transposon insertion site, hybridisation to a probe on the oligonucleotide array will be observed even though that probe corresponds to an essential gene. However, that probe will show no hybridisation with the other pool of RNA target sequences which comprise the flanking sequence from the other side of the transposon. Thus, in this type of TMDH, a probe in the oligonucleotide array to which at least one of the pools of RNA target sequences does not hybridise is likely to correspond to a gene that has not been disrupted by a transposon and may therefore be assigned as an essential gene.

In the methods described above (and also in those described below for the identification of conditional essential genes), the pools of RNA sequences may be directly hybridised to arrays as set out above. Alternatively, before hybridisation is carried out, the pools of RNA sequences may be subjected to reverse transcription to generate pools of cDNAs. The sequence of the particular transposon of the invention used to generate the library of mutants may be used to design oligonucleotide primers suitable for priming reverse transcription or, alternatively, random primers may be used. A label may be included in the reverse transcription reaction so that labelled cDNA pools are generated. The cDNA pools may be then be hybridised to arrays as is described above in relation to RNA pools.

The TMDH methods described above may also be used for the identification of conditional essential genes. Conditional essential genes are those which are not absolutely essential for bacterial survival, but are essential for survival in particular environments e.g. for growth/proliferation, in a host (in the case of a pathogenic bacterium) or for survival at elevated temperatures. Such environments are described here as conditional restraints.

In order to isolate conditional essential genes, a library of transposon mutants is generated under control conditions (eg. growth at 37° C. in complete media). The library of mutants is then subjected to some conditional restraint. For example, the library of mutants can be inoculated in a suitable host, if it is a pathogen. Alternatively, the library of mutants can be grown at an elevated temperature. After the library of mutants has been subjected to the conditional restraint it can be recovered.

The library of mutants may be recovered by, for example, recovering tissue such as the liver and/or spleen from a host and plating out an extract derived from that tissue on growth medium. All surviving colonies on the plate represent mutants where a transposon insertion has occurred in a non-essential sequence. The surviving colonies can be pooled to give an output library of mutants which can then be subjected to one of the TMDH methods set out above.

Alternatively, non-essential DNA sequences may be recovered directly from tissue (such as the liver and/or spleen) of an infected host without an intervening step of plating out an extract of the isolated tissue. In this regard, the presence of one, or preferably two, homing endonuclease recognition sites in the transposon is crucial. Thus, the tissue may be subjected to digestion with a homing endonuclease for which there is a corresponding recognition site in the relevant transposon. Typically, the tissue would be homogenised before digestion. Transposon ends (i.e. transposon:flanking sequence fragments) may then be recovered from the tissue. This may be achieved by annealing biotinylated linkers to the transposon ends and recovering the resulting biotinylated transposon ends with, for example, strepatavidin-coated beads. The isolated transposon:flanking sequence fragments may then be digested with a restriction endonuclease which cuts in the sequence flanking the transposon insertion site. Alternatively, the fragments may be digested with a restriction endonuclease prior to recovery from the tissue.

The library of mutants that have been exposed to the conditional restraint will lack mutants which carry transposons in those genes essential for growth under the conditional environment, for example growth/proliferation in a host organism.

The control and conditional restraint libraries can then be subjected to the TMDH protocols described above using the a transposon of the invention. Clearly, if transposon:flanking sequence fragments have been isolated directly, for example from a host tissue using capture of, those fragments enter a TMDH protocol at the stage of preparing RNA by transcription from the RNA polymerase site(s).

It is not necessary to use the same TMDH protocol for each library. The two resulting RNA target sequence libraries may then be hybridised separately to high density oligonucleotide arrays. Alternatively they can be hybridised to the same array, if the control and conditional restraint libraries are differentially labelled for example.

Comparison of the results given with the control and the conditional restraint libraries will allow the identification of genes which permit survival in the conditional restraint. Genes identified as essential for survival in the conditional restraint library, but not identified as essential for survival under control conditions should represent genes that are essential for survival under the conditional restraint conditions. In particular, probes which show hybridisation with RNA target sequences from the input library but which show no hybridisation or substantially no hybridisation (there may be a low level of background non-specific hybridisation) with RNA target sequences from the output library are strong candidates for sequences corresponding to conditional essential genes. The same “bleed through” considerations apply as set out above and the modified TNDH protocols for overcoming such “bleed through” may need to be used.

In the case of the analysis of conditional mutations in a pathogen, a library of Salmonella typhimurium transposon mutants, for example, can be used to infect a mouse. Following infection, bacteria target to livers and spleens and the course of infection can be conveniently followed by performing viable bacterial counts on those organs. The bacteria recovered from the livers and spleens can be grown on suitable plates. In the case of the conditional restraint at elevated temperature, a transposon-tagged library can be grown at 42° C.

Other conditional restraints include growth of antibiotic resistant bacteria in the presence of antibiotics. This may reveal genes which are essential for antibiotic resistance. Such genes would be targets for drugs with the ability to lower bacterial resistance to particular antibiotics. Organisms could be grown in the presence of carcinogens, UV or other agents that cause oxidative stress and thus genes that confer resistance to growth under those conditions may be identified.

Potential essential gene sequences and conditional essential gene sequences identified by a TMDH strategy using a transposon of the invention may be verified using a method based on allelic exchange. This technique is particularly suitable for analysis of bacterial genes. PCR primers can be used to generate left- and right-arm sequences corresponding to the target gene sequence and ligated with a kanamycin-resistance encoding gene cassette. The resulting cassette can be introduced into a suicide vector, for example a plasmid-based vector, which is unable to replicate in a host bacterium.

In the case of a candidate essential gene, the resulting construct can be introduced into the bacterial strain from which the candidate gene originates. If the target gene is essential, it should be impossible to isolate allelic-exchange mutants that have a disrupted version of the target gene. In the case of a candidate conditional essential gene, the essential gene can be introduced into the bacterial strain from which the candidate gene originates. Allelic-exchange mutants can be isolated and subjected to growth under the conditional restraint. If the candidate gene is a conditional essential gene, it should not be possible for the allelic-exchange mutants to survive under the conditional restraint.

Similar experiments may be performed for other organisms.

The use of bioinformatics may allow the rapid isolation of further essential and conditional essential genes. A gene identified by TMDH using a transposon of the invention may be used to search databases containing sequence information from other species in order to identify orthologous genes from those species. Genes so identified can be tested for being essential or conditionally essential using the genetic techniques described above. For example, an E. coli gene is identified as essential using a method as described above. This may allow the identification of a putative orthologue from Salmonella. That Salmonella gene may be tested by allelic exchange and the construction of conditional mutants in Salmonella as described above. Further orthologues may be identified in more distantly related organisms, for example from Plasmodium species.

Suitable bioinformatics programs are well known to those skilled in the art. For example, the Basic Local Alignment Search Tool (BLAST) program (Altschul et al., 1990, J. Mol. Biol. 215, 403-410. and Altschul et al., 1997, Nucl. Acids Res. 25, 3389-3402.) may be used. Suitable databases for searching are for example, EMBL, GENBANK, TIGR, EBI, SWISS-PROT and trEMBL.

Organisms that may be used in the invention are those for which it is possible to carry out transposon mutagenesis and thus, those that can give rise to a library of transposon mutants. Clearly, if the genome is bigger, more mutants will have to be produced in order to give a better chance of achieving saturation mutagenesis. Suitable organisms include prokaryotic and eukaryotic organisms. Suitable prokaryotes include bacteria Preferred bacteria are those which are animal or human or plant pathogens.

The bacteria used may be Gram-negative or Gram-positive. The bacteria may be for example, from the genera Escherichia, Salmonella, Vibrio, Haemophilus, Neisseria, Yersinia, Bordetella, Brucella, Shigella, Klebsiella, Enterobacter, Serracia, Proteus, Vibrio, Aeromonas, Pseudomonas, Acinetobacter, Moraxella, Flavobacterium, Actinobacillus, Staphylococcus, Streptococcus, Mycobacterium, Listeria, Clostridium, Pasteurella, Helicobacter, Campylobacter, Lawsonia, Mycoplasma, Bacillus, Agrobacterium, Rhizobium, Erwinia or Xanthomonas. Examples of some of the above mentioned genera are Escherichia coli—a cause of diarrhoea in humans; Salmonella typhimurium—the cause of salmonellosis in several animal species; Salmonella typhi—the cause of human typhoid fever; Salmonella enteritidis—a cause of food poisoning in humans; Salmonella choleraesuis—a cause of salmonellosis in pigs; Salmonella dublin—a cause of both a systemic and diarrhoeal disease in cattle, especially of new-born calves; Haemophilus influenzae—a cause of meningitis; Neisseria gonorrhoeae—a cause of gonorrhoea; Yersinia enterocolitica—the cause of a spectrum of diseases in humans ranging from gastroenteritis to fatal septicemic disease; Bordetella pertussis—the cause of whooping cough; Brucella abortus—a cause of abortion and infertility in cattle and a condition known as undulant fever in humans; Vibrio cholerae—a cause of cholera, Clostridium tetani—a cause of tetanus; Bacillus anthracis—a cause of anthrax. Suitable eukaryotes include fungi, plants and animals. Preferred eukaryotes include animal or human parasites and plant pests.

Suitable fungi include the animal pathogens including Candida albicans—a cause of thrush, Trichophyton spp.—a cause of ringworm in children, athlete's foot in adults. Other suitable fungi include the plant pathogens Phytophthora infestans, Plasmopara viticola, Peronospora spp., Saprolegnia spp., Erysiphe spp., Ceratocystis ulmi, Monilinia fructigena, Venturia inequalis, Claviceps purpurea, Diplocarpon rosae, Puccinia graminis, Ustilago avenae.

Suitable animal parasites include Plasmodium spp., Trypanasoma spp., Giarda spp., Trichomonas spp. and Schistosoma spp. Other animal parasites include the various platyhelminth, nematode and annelid parasites.

Suitable plant pests include insects, nematodes and molluscs such as slugs and snails.

Suitable plants include monocotyledons and dicotyledons.

Preferred organisms are those for which the entire genome is known and for which it may be possible to construct a high density oligonucleotide array covering the entire genome or all of the open reading frames.

Essential and conditional essential genes, particularly essential genes are targets for drug discovery. That is, essential and conditional essential genes of bacteria and the polypeptides which they encode may represent targets for antibacterial substances, for example. Similarly essential and conditional essential genes of fungi and eukaryotic parasites, pests and plants and the proteins which they encode may represent targets for fungicides, antiparasitics, pesticides and herbicides respectively. Fungicides may have both animal and plant applications. Additionally, conditional essential genes may represent targets for the generation of attenuated vaccines, particularly in the case of bacterial conditional essential genes.

Furthermore, if a particular gene is essential or conditionally essential for a number of different bacteria, fungi, parasites, pests or plants, that gene and the polypeptide it encodes may represent a target for substances with a broad-spectrum of activity.

An essential or conditional essential gene identified by one of the methods described above using a transposon of the invention and the polypeptide which it encodes may be used in a method for identifying an inhibitor of transcription and/or translation of the gene and/or activity of the polypeptide encoded by the gene. Such a method may comprise identifying an essential or conditional essential gene using a method of the invention and then determining whether a test substance inhibits the transcription and/or translation of a gene thus identified or inhibits the activity of a polypeptide encoded by such a gene.

Such a substance may be referred to as an inhibitor of an essential or conditional essential gene. Thus, an inhibitor of an essential or conditional essential gene is a substance which inhibits expression and/or translation of that essential gene and/or activity of the polypeptide encoded by that essential or conditional essential gene.

Any suitable assay may be carried out to determine whether a test substance is an inhibitor of an essential or conditional essential gene. For example, the promoter of an essential or conditional essential gene may be linked to a coding sequence for a reporter polypeptide. Such a construct may be contacted with a test substance under conditions in which, in the absence of the test substance expression of the reporter polypeptide would occur. This would allow the effect of the test substance on expression of the essential or conditional essential gene to be determined.

Substances which inhibit translation of an essential or conditional essential gene may be isolated, for example, by contacting the mRNA of the essential or conditional essential gene with a test substance under conditions that would permit translation of the mRNA in the absence of the test substance. This would allow the effect of the test substance on translation of the essential or conditional essential gene to be determined.

Substances which inhibit activity of a polypeptide encoded by the essential gene may be isolated, for example, by contacting the polypeptide with a substrate for the polypeptide and a test substance under conditions that would permit activity of the polypeptide in the absence of the test substance. This would allow the effect of the test substance on activity of the polypeptide encoded by the essential or conditional essential gene to be determined.

Suitable control experiments can be carried out. For example, a putative inhibitor should be tested for its activity against other promoters, mRNAs or polypeptides to discount the possibility that it is a general inhibitor of gene transcription, translation or polypeptide activity.

Suitable test products which can be tested in the above assays include combinatorial libraries, defined chemical entities, peptide and peptide mimetics, oligonucleotides and natural product libraries, such as display (e.g. phage display libraries) and antibody products. Antibody products include monoclonal and polyclonal antibodies, single chain antibodies, chimaeric antibodies and CDR-grafted antibodies.

Typically, organic molecules will be screened, preferably small organic molecules which have a molecular weight of from 50 to 2500 daltons. Candidate products can be biomolecules including, saccharides, fatty acids, steroids, purines, pyrimidines, derivatives, structural analogs or combinations thereof. Candidate agents are obtained from a wide variety of sources including libraries of synthetic or natural compounds. Known pharmacological agents may be subjected to directed or random chemical modifications, such as acylation, alkylation, esterification, amidification, etc. to produce structural analogs.

Test substances may be used in an initial screen of, for example, for example from 10 to 100 substances per reaction, and the substances of these batches which show inhibition or stimulation tested individually. Test substances may be used at a concentration of from 1 nM to 1000 μM, preferably from 1 μM to 100 μM, more preferably from 1 μM to 10 μM. Suitable test substances for inhibitors of essential or conditional essential genes include combinatorial libraries, defined chemical entities, peptides and peptide mimetics, oligonucleotides and natural product libraries.

The test substances may be used in an initial screen of, for example, ten substances per reaction, and the substances of batches which show inhibition tested individually.

An inhibitor of an essential or conditional essential gene is one which inhibits expression and/or translation of that essential gene and/or activity of the polypeptide encoded by that essential or conditional gene. Preferred inhibitors of the invention are those which inhibit essential gene expression and/or translation and/or activity by at least 10%, at least 20%, at least 30%, at least 40%, at least 50%, at least 60%, at least 70%, at least 80%, at least 90%, at least 95% or at least 99% at a concentration of the inhibitor of 1 ngml⁻¹, 10 ngml⁻¹, 100 ngml⁻¹, 500 ngml⁻¹, 1 μgml⁻¹, 10 μgm⁻¹, 100 μgml⁻¹, 500 μgml⁻¹, 1 mgml⁻¹, 10 mgml⁻¹, 100 mgml⁻¹. The percentage inhibition represents the percentage decrease in expression and/or translation and/or activity in a comparison of assays in the presence and absence of the test substance. Any combination of the above mentioned degrees of percentage inhibition and concentration of inhibitor may be used to define an inhibitor of the invention, with greater inhibition at lower concentrations being preferred.

Test substances which show activity in assays such as those described above can be tested in in vivo systems, such as an animal model of infection for antibacterial activity or a plant model for herbicidal activity. Thus, candidate inhibitors could be tested for their ability to attenuate bacterial infections in mice in the case of an antibacterial or for their ability to inhibit growth of plants in the case of a herbicide.

Inhibitors of bacterial, fungal or eukaryotic parasite essential or conditional essential genes may be used in a method of treatment of the human or animal body by therapy. In particular such substances may be used in a method of treatment of a bacterial, fungal or eukaryotic parasite infection. Such substances may also be used for the manufacture of a medicament for use in the treatment of a bacterial, fungal or eukaryotic parasite infections The condition of a patient suffering from such an infection can be improved by administration of an inhibitor. A therapeutically effective amount of an inhibitor may be given to a human patient in need thereof. Inhibitors of bacterial, fungal or eukaryotic parasite essential or conditional essential genes may be administered in a variety of dosage forms. Thus, they can be administered orally, for example as tablets, troches, lozenges, aqueous or oily suspensions, dispersible powders or granules. The inhibitors may also be administered parenterally, either subcutaneously, intravenously, intramuscularly, intrasternally, transdermally or by infusion techniques. The inhibitors may also be administered as suppositories. A physician will be able to determine the required route of administration for each particular patient.

The formulation of an inhibitor for use in preventing or treating a bacterial or fungal infection will depend upon factors such as the nature of the exact inhibitor, whether a pharmaceutical or veterinary use is intended, etc. An inhibitor may be formulated for simultaneous, separate or sequential use.

An inhibitor is typically formulated for administration in the present invention with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

A therapeutically effective amount of an inhibitor is administered to a patient. The dose of an inhibitor may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg of body weight, according to the activity of the specific inhibitor, the age, weight and conditions of the subject to be treated, the type and severity of the degeneration and the frequency and route of administration. Preferably, daily dosage levels are from 5 mg to 2 g.

Conditional essential genes are good candidates for use in the preparation of live attenuated vaccines. The principle behind vaccination is to induce an immune response in the host thus providing protection against subsequent challenge with a pathogen. This may be achieved by inoculation with a live attenuated strain of the pathogen, i.e. a strain having reduced virulence such that it does not cause the disease caused by the virulent pathogen. Bacteria which carry mutations in conditional essential genes required for survival (i.e. growth/proliferation) in a host isolated according to the methods described above may be good candidates for use in live attenuated vaccines.

Mutations introduced into a bacterium for use in a vaccine generally knock-out the function of the conditional essential gene, for example a gene required for growth/proliferation in a host, completely. This may be achieved either by abolishing synthesis of any polypeptide at all from the gene or by making a mutation that results in synthesis of non-functional polypeptide. In order to abolish synthesis of polypeptide, either the entire gene or its 5′-end may be deleted. A deletion or insertion within the coding sequence of a gene may be used to create a gene that synthesises only non-functional polypeptide (e.g. polypeptide that contains only the N-terminal sequence of the wild-type protein).

The bacterium may have mutations in one or more, for example two, three or four conditional essential genes. The mutations are non-reverting mutations. These are mutations that show essentially no reversion back to the wild-type when the bacterium is used as a vaccine. Such mutations include insertions and deletions. Insertions and deletions are preferably large, typically at least 10 nucleotides in length, for example from 10 to 600 nucleotides. Preferably, the whole coding sequence is deleted.

The bacterium used in the vaccine preferably contains only defined mutations, i.e. mutations which are characterised. It is clearly undesirable to use a bacterium which has uncharacterised mutations in its genome as a vaccine because there would be a risk that the uncharacterised mutations may confer properties on the bacterium that cause undesirable side-effects.

The attenuating mutations may be introduced by methods well known to those skilled in the art. Appropriate methods include cloning the DNA sequence of the wild-type gene into a vector, e.g. a plasmid, and inserting a selectable marker into the cloned DNA sequence or deleting a part of the DNA sequence, resulting in its inactivation. A deletion may be introduced by, for example, cutting the DNA sequence using restriction enzymes that cut at two points in or just outside the coding sequence and ligating together the two ends in the remaining sequence with an antibiotic resistance determinant. A plasmid carrying the inactivated DNA sequence can be transformed into the bacterium by known techniques such as electroporation or conjugation for example. It is then possible by suitable selection to identify a mutant wherein the inactivated DNA sequence has recombined into the chromosome of the bacterium and the wild-type DNA sequence has been rendered non-functional by homologous recombination.

The attenuated bacterium of the invention may be genetically engineered to express an antigen that is not expressed by the native bacterium (a “heterologous antigen”), so that the attenuated bacterium acts as a carrier of the heterologous antigen. The antigen may be from another organism, so that the vaccine provides protection against the other organism. A multivalent vaccine may be produced which not only provides immunity against the virulent parent of the attenuated bacterium but also provides immunity against the other organism. Furthermore, the attenuated bacterium may be engineered to express more than one heterologous antigen, in which case the heterologous antigens may be from the same or different organisms. The heterologous antigen may be a complete protein or a part of a protein containing an epitope. The antigen may be from a virus, prokaryote or a eulcaryote, for example another bacterium, a yeast, a fungus or a eukaryotic parasite. The antigen may be from an extracellular or intracellular protein. More especially, the antigenic sequence may be from E. coli, tetanus, hepatitis A, B or C virus, human rhinovirus such as type 2 or type 14, herpes simplex virus, poliovirus type 2 or 3, foot-and-mouth disease virus, influenza virus, coxsackie virus or Chlamydia trachomatis. Useful antigens include non-toxic components of E. coli heat labile toxin, E.coli K88 antigens, ETEC colonization factor antigens, P.69 protein from B. pertussis and tetanus toxin fragment C.

The DNA encoding the heterologous antigen is expressed from a promoter that is active in vivo. Two promoters that have been shown to work well in Salmonella are the nirB promoter and the htrA promoter. For expression of the ETEC colonization factor antigens, the wild-type promoters could be used. A DNA construct comprising the promoter operably linked to DNA encoding the heterologous antigen may be made and transformed into the attenuated bacterium using conventional techniques. Transformants containing the DNA construct may be selected, for example by screening for a selectable marker on the construct. Bacteria containing the construct may be grown in vitro before being formulated for administration to the host for vaccination purposes.

The vaccine may be formulated using known techniques for formulating attenuated bacterial vaccines. The vaccine is advantageously presented for oral administration, for example in a lyophilised encapsulated form. Such capsules may be provided with an enteric coating comprising, for example, Eudragate “S” (Trade Mark), Eudragate “L” (Trade Mark), cellulose acetate, cellulose phthalate or hydroxypropylmethyl cellulose. These capsules may be used as such, or alternatively, the lyophilised material may be reconstituted prior to administration, e.g. as a suspension. Reconstitution is advantageously effected in a buffer at a suitable pH to ensure the viability of the bacteria In order to protect the attenuated bacteria and the vaccine from gastric acidity, a sodium bicarbonate preparation is advantageously administered before each administration of the vaccine. Alternatively, the vaccine may be prepared for parenteral administration, intranasal administration or intramuscular administration.

The vaccine may be used in the vaccination of a mammalian host, particularly a human host but also an animal host. An infection caused by a microorganism, especially a pathogen, may therefore be prevented by administering an effective dose of a vaccine prepared according to the invention. The dosage employed will ultimately be at the discretion of the physician, but will be dependent on various factors including the size and weight of the host and the type of vaccine formulated. However, a dosage comprising the oral administration of from 10⁷ to 10¹¹ bacteria per dose may be convenient for a 70 kg adult human host.

Inhibitors of bacterial, fungal and pest essential or conditional essential genes may be administered to plants in order to prevent or treat bacterial, fungal or pest infections; the term pest includes any animal which attacks a plant. Thus inhibitors of the invention may be useful as pesticides. Inhibitors of plant essential or conditional essential genes may be administered to plants in order to reduce or stop plant growth, that is to act as a herbicide.

The inhibitors of the present invention are normally applied in the form of compositions together with one or more agriculturally acceptable carriers or diluents and can be applied to the crop area or plant to be treated, simultaneously or in succession with further compounds.

The inhibitors of the invention can be selective herbicides, bacteriocides, fungicides or pesticides or mixtures of several of these preparations, if desired together with further carriers, surfactants or application-promoting adjuvants customarily employed in the art of formulation. Suitable carriers and diluents correspond to substances ordinarily employed in formulation technology, e.g. natural or regenerated mineral substances, solvents, dispersants, wetting agents, tackifiers, binders or fertilizers.

A preferred method of applying active ingredients of the present invention or an agrochemical composition which contains at least one of the active ingredients is leaf application. The number of applications and the rate of application depend on the intensity of infestation by the pathogen. However, the active ingredients can also penetrate the plant through the roots via the soil (systemic action) by impregnating the locus of the plant with a liquid composition, or by applying the compounds in solid form to the soil, e.g. in granular form (soil application). The active ingredients may also be applied to seeds (coating) by impregnating the seeds either with a liquid formulation containing active ingredients, or coating them with a solid formulation. In special cases, further types of application are also possible, for example, selective treatment of the plant stems or buds.

The active ingredients are used in unmodified form or, preferably, together with the adjuvants conventionally employed in the art of formulation, and are therefore formulated in known manner to emulsifiable concentrates, coatable pastes, directly sprayable or dilutable solutions, dilute emulsions, wettable powders, soluble powders, dusts, granulates, and also encapsulations, for example, in polymer substances. Like the nature of the compositions, the methods of application, such as spraying, atomizing, dusting, scattering or pouring, are chosen in accordance with the intended objectives and the prevailing circumstances. Advantageous rates of application are normally from 50 g to 5 kg of active ingredient (a.i.) per hectare (“ha”, approximately 2.471 acres), preferably from 100 g to 2 kg a.i./ha, most preferably from 200 g to 500 g a.i./ha.

The formulations, compositions or preparations containing the active ingredients and, where appropriate, a solid or liquid adjuvant, are prepared in known manner, for example by homogeneously mixing and/or grinding active ingredients with extenders, for example solvents, solid carriers and, where appropriate, surface-active compounds (surfactants).

Suitable solvents include aromatic hydrocarbons, preferably the fractions having 8 to 12 carbon atoms, for example, xylene mixtures or substituted naphthalene, phthalate such as dibutyl phthalate or dioctyl phthalate, aliphatic hydrocarbons such as cyclohexane or paraffins, alcohols and glycols and their ethers and esters, such as ethanol, ethylene glycol, monomethyl or monoethyl ether, ketones such as cyclohexanone, strongly polar solvents such as N-methyl-2-pyrrolidone, dimethyl sulfoxide or dimethyl formamide, as well as epoxidized vegetable oils such as epoxidized coconut oil or soybean oil; or water.

The solid carriers used e.g. for dusts and dispersible powders, are normally natural mineral fillers such as calcite, talcum, kaolin, montmorillonite or attapulgite. In order to improve the physical properties it is also possible to add highly dispersed silicic acid or highly dispersed absorbent polymers. Suitable granulated adsorptive carriers are porous types, for example pumice, broken brick, sepiolite or bentonite; and suitable nonsorbent carriers are materials such as calcite or sand. In addition, a great number of pregranulated materials of inorganic or organic nature can be used, e.g. especially dolomite or pulverized plant residues.

Depending on the nature of the active ingredient to be used in the formulation, suitable surface-active compounds are nonionic, cationic and/or anionic surfactants having good emulsifying, dispersing and wetting properties. The term “surfactants” will also be understood as comprising mixtures of surfactants. Suitable anionic surfactants can be both water-soluble soaps and water-soluble synthetic surface-active compounds.

Suitable soaps are the alkali metal salts, alkaline earth metal salts or unsubstituted or substituted ammonium salts of higher fatty acids (chains of 10 to 22 carbon atoms), for example the sodium or potassium salts of oleic or stearic acid, or of natural fatty acid mixtures which can be obtained for example from coconut oil or tallow oil. The fatty acid methyltaurin salts may also be used.

More frequently, however, so-called synthetic surfactants are used, especially fatty sulfonates, fatty sulfates, sulfonated benzimidazole derivatives or alkylarylsulfonates.

The fatty sulfonates or sulfates are usually in the form of alkali metal salts, alkaline earth metal salts or unsubstituted or substituted ammoniums salts and have a 8 to 22 carbon alkyl radical which also includes the alkyl moiety of alkyl radicals, for example, the sodium or calcium salt of lignonsulfonic acid, of dodecylsulfate or of a mixture of fatty alcohol sulfates obtained from natural fatty acids. These compounds also comprise the salts of sulfuric acid esters and sulfonic acids of fatty alcohol/ethylene oxide adducts. The sulfonated benzimidazole derivatives preferably contain 2 sulfonic acid groups and one fatty acid radical containing 8 to 22 carbon atoms. Examples of alkylarylsulfonates are the sodium, calcium or triethanolamine salts of dodecylbenzenesulfonic acid, dibutylnaphthalenesulfonic acid, or of a naphthalenesulfonic acid/formaldehyde condensation product. Also suitable are corresponding phosphates, e.g. salts of the phosphoric acid ester of an adduct of p-nonylphenol with 4 to 14 moles of ethylene oxide.

Non-ionic surfactants are preferably polyglycol ether derivatives of aliphatic or cycloaliphatic alcohols, or saturated or unsaturated fatty acids and alkylphenols, said derivatives containing 3 to 30 glycol ether groups and 8 to 20 carbon atoms in the (aliphatic) hydrocarbon moiety and 6 to 18 carbon atoms in the alkyl moiety of the alkylphenols.

Further suitable non-ionic surfactants are the water-soluble adducts of polyethylene oxide with polypropylene glycol, ethylenediamine propylene glycol and alkylpolypropylene glycol containing 1 to 10 carbon atoms in the alkyl chain, which adducts contain 20 to 250 ethylene glycol ether groups and 10 to 100 propylene glycol ether groups. These compounds usually contain 1 to 5 ethylene glycol units per propylene glycol unit.

Representative examples of non-ionic surfactants are nonylphenolpolyethoxyethanols, castor oil polyglycol ethers, polypropylene/polyethylene oxide adducts, tributylphenoxypolyethoxyethanol, polyethylene glycol and octylphenoxyethoxyethanol. Fatty acid esters of polyoxyethylene sorbitan and polyoxyethylene sorbitan trioleate are also suitable non-ionic surfactants.

Cationic surfactants are preferably quaternary ammonium salts which have, as N-substituent, at least one C₈-C₂₂ alkyl radical and, as further substituents, lower unsubstituted or halogenated alkyl, benzyl or lower hydroxyalkyl radicals. The salts are preferably in the form of halides, methylsulfates or ethylsulfates, e.g. stearyltrimethylammonium chloride or benzyldi(2-chloroethyl)ethylammonium bromide.

The surfactants customarily employed in the art of formulation are described, for example, in “McCutcheon's Detergents and Emulsifiers Annual”, MC Publishing Corp. Ringwood, N.J., 1979, and Sisely and Wood, “Encyclopaedia of Surface Active Agents,” Chemical Publishing Co., Inc. New York, 1980. The agrochemical compositions usually contain from about 0.1 to about 99% preferably about 0.1 to about 95%, and most preferably from about 3 to about 90% of the active ingredient, from about 1 to about 99.9%, preferably from about 1 to 99%, and most preferably from about 5 to about 95% of a solid or liquid adjuvant, and from about 0 to about 25%, preferably about 0.1 to about 25%, and most preferably from about 0.1 to about 20% of a surfactant.

Whereas commercial products are preferably formulated as concentrates, the end user will normally employ dilute formulations.

The following Examples illustrate the invention:

### EXAMPLES

Materials and Methods

Unless indicated otherwise, the methods used are standard biochemical techniques. Examples of suitable general methodology textbooks include Sambrook et al., Molecular Cloning, a Laboratory Manual (1989) and Ausubel et al., Current Protocols in Molecular Biology (1995), John Wiley & Sons, Inc.

E. coli strains were grown on L-Agar (Sigma) and in L-Broth (Sigma). Kanamycin was purchased from Sigma S. typhimurium SL1344 was grown in Tryptic Soy Broth (TSB, Oxoid).

### Example 1

### Construction of a Gene-Kelly Transposon

The EZ:Tn R6k ori Kan transposon (Epicentre) was used as a PCR template with oligonucleotides:

(SEQ ID NO: 2)

| | |
|---|---|
| 97 | 5′-CAGCTGTCTCTTATACACATCTCCCTATAGTGAGTCGTATTAC |
| CCATAATACCCATAATAGCTGTTTGCCAgtcgactctagagg-3′; | |
| and | |

(SEQ ID NO: 3)

| | |
|---|---|
| 97 | 5′-CAGCTGTCTCTTATACACATCTCTTCTATAGTGTCACCTAAATA |
| GGGATAACAGGGTAATGaattcgttaatacagatgt-3′. | |

ME are italicised, the RNA polymerase binding sites are in bold, and the homing endonuclease sites are underlined. These oligonucleotides incorporate the T7 RNA polymerase site with the homing endonuclease site PI-PspI, and the SP6 RNA polymerase site with the homing endonuclease site for I-SceI, respectively. PCR was carried out according to protocol for Roche Expand Hi-fidelity Kit.

A first round of PCR, to introduce the homing endonuclease and RNA polymerase sites, was carried out with an initial denaturation step at 96° C. for 3 min, then 5 cycles of 96° C. 30s, 25° C. 90s, 72° C. 2 min 30s, then a further 25 cycles at 96° C. 30s, 50° C. 90s, 72° C. 2 min 30s, final extension of 4 minutes and then cooled to 4° C. The PCR product was separated by gel electrophoresis, and the band (corresponding to the expected product at 2044 bp) cut out and Gel extracted according to the Qiagen gel extraction protocol. The PCR products were re-ligated (according to Gibco protocol for T4 DNA ligase) overnight at 16° C. The ligation mixture was then purified using the Qiagen Gel Extraction protocol and the circularised transposon eluted in 50 μl water. 1 μl of the Transposon DNA was then electroporated into 40 μl TransforMax™ EC100D™ pir⁺ Electrocompetent E. coli (Epicentre; using 0.1 cm cuvettes at 200Ω, 25 μF and 20 KV/cm), outgrowth was in 1 ml SOC medium (Gibco life technologies) for 1 h at 37° C. The transformation was then plated out on L-Agar (Sigma) plates containing Kanamycin at 30 μg/ml and incubated overnight at 37° C. 12 colonies were then picked and grown up overnight in LB-Kan 30 μg/ml and 1.5 ml of culture was used for DNA-mini-preps according to the Qiagen protocol. The DNA sequence of the resulting constructs was then determined on a Beckman CEQ DNA sequencer, (using manufacturers recommended conditions) with kan-2-FP-1 forward primer (Epicentre) as a DNA sequencing primer. The sequence of clone 11(GK11) was found to contain a single base pair change in one of the ME sequences. To correct this sequence change, an experiment using a second round of PCR was designed with an oligonucleotide that corrected the single base pair change. The clone GK11 (harbouring a single-point mutation in the ME) was re-PCR'd using un-phosphorylated ME primers (96° C. for 3 min, followed by 30 cycles of 96° C. for 30s, 45° C. for 90s, 72° C. for 150s). The PCR products were then cloned into pBAD-TOPO (Invitrogen) and transformed into E. coli TOP10 chemically competent cells according to the Invitrogen protocol. The transformation mix was plated out on L-Agar containing kanamycin at 30 μg/ml and incubated overnight at 37° C. 6 colonies were then picked and grown in L-Broth containing kanamycin at 30 μg/ml overnight at 37° C. DNA from 1.5 mL samples of these cultures was then prepared using the miniprep method (Qiagen QIAprep spin miniprep kit) according to the Qiagen protocol. The DNA sequences of the inserts from each of these 6 minipreps was then determined with oligonucleotides kan-2-FP-1 and R6kan-2-RP-1 (Epicentre) as sequencing primers according to Beckman CEQ protocol. One of the isolates, clone 6A was correct when sequenced, named pBAD-GK6A, and was selected for further study.

### Example 2

### Evaluation of the Gene-Kelly Transposon

(i) T7 and SP6 RNA Polymerase Evaluation

In order to evaluate the novel transposon generated in Example 1, 0.2 μg of pBAD-GK6A was used as template in an in vitro transcription (TVT) reactions using both SP6 (SP6 Megascript kit; Ambion) and T7 (T7 Megashortscipt kit, Ambion) RNA polymerases, according to Ambion protocols. RNA produced was purified using the Qiagen RNeasy mini Kit, and the amount of RNA produced measured at A₂₆₀. Transcription was observed both with the SP6 and T7 IVT reactions.

(ii) Restriction Digest of Transposon DNA with Homing Endonucleases

In order to determine whether or not the homing endonuclease sites were functional in the GK transposon, plasmid DNA (0.5 μg) was digested with I-SceI and PI-PspI. Separation of the resulting DNA products by agarose gel electrophoresis showed a single linearised band of the correct size.

(iii) Transposition of Salmonella typhimurium

pBAD-GK6A (0.1 μg) was electroporated into 40 μl S. typhimurium SL1344 electrocompetent cells (SL1344 was grown to an OD 0.5 in 100 ml tryptic-soy broth (TSB from Oxoid) at 37° C. Cells were centrifuged at 5000×g for 10 min at 4° C., washed three times in 50 ml 10% glycerol before a final re-suspension in 1 ml of 10% glycerol) using a 0.2 cm cuvette, 2000, 25 μF and 12 kV/cm, outgrowth was in 1 ml SOC (Gibco) at 37° C. for 1 h, the transformation was then plated onto L-Agar plates containing Kanamycin at 50 μg/ml plates and grown overnight at 37° C. A colony was then picked and grown in 2.5 litres L-Broth containing Kanamycin at 50 μg/ml overnight at 37° C. A Qiagen Qiafilter Mega plasmid kit was used to purify pBAD-GK6A.

### Example 3

### Generation of S. typhimurium SL1344 Mutants with Transposon GK6a

The transposome complex was generated from pBAD-GK6A as follows. pBAD-GK6a (100 μg) of was digested with XmnI and NcoI (NEB) in NEB buffer 2 (with 1 μg BSA/ml), overnight at 37° C. The entire digest was then run out on a 0.8% agarose gel, the ˜2 kb band, the GK transposon, was gel extracted using the Qiagen gel extraction kit, and eluted from 1 spin column in 50 μl TE pH8.5. The GK transposome complex was generated according to Epicentre protocols and electroporated into electrocompetent S. typhimurium SL1344 cells as described above. Following outgrowth the cells were subsequently plated out on Tryptic Soy Agar plates (TSA, Oxoid) containing Kanamycin at 50 μg/ml and incubated overnight at 37° C. A total of 480 mutants were picked and grown overnight in 2 ml TSB (Oxoid) containing Kanamycin at 50 μg/ml and glycerol stocks made (20% glycerol TSB).

### Example 4

### Recovery of Transposon for Sequencing (use of R6k Origin of Replication)

Transposons and adjacent flanking DNA, corresponding to genes that have been disrupted by transposon insertion can be recovered from mutant chromosomal DNA samples using the use of R6k origin of replication. Digestion of chromosomal DNA purified from S. typhimurium SL1344 GK mutants with a restriction enzyme that does not cut in the transposon, followed by circularising the fragments, and transformation into a pir⁺ strain of E. coli, results in the “rescuing” of this DNA.

Mutants 1-50 were grown up individually in 2 ml TSB containing Kanamycin at 50 μg/ml overnight at 37° C. Samples (1.5 ml) was used to prepare chromosomal DNA using the Qiagen DNeasy tissue kit. A total of 5 μl (0.5 μg) of each of the fifty chromosomal preps was digested with EcoRV (NEB) in a final volume of 20 μl, overnight at 37° C. The EcoRV was heat inactivated at 80° C. for 20 minutes. The 20 μl digest was then religated in 100 μl final volume using Gibco T4 DNA ligase, 48 hours at 4° C. Each religation was then individually cleaned up using a Qiagen gel extraction spin column and eluted in 50 μl water. Ligations (4 μl, 0.04 μg) were electroporated into electrocompetent pir⁺ E. coli (EC100D, Epicentre) according to Epicentre protocols and plated on L-Agar containing Kanamycin at 30 μg/ml and incubated overnight at 37° C. Colonies were obtained from 46 of the electroporations, and were subsequently grown up in 5 ml L-Broth containing Kanamycin at 30 μg/ml 37° C. overnight Plasmid DNA (2 μg) from these clones was sequenced according to the Beckman CEQ protocol using oligonucleotide 108 as a sequencing primer (T7 end of the transposon).

### Example 5

### Generation of RNA Run-Offs Using iPCR and IVT for Target Hybridisation to Microarrays

The generation of labelled target from GK mutants can be achieved by inverse PCR (iPCR) amplification of each end of the transposon followed by IVT reactions using either SP6 or T7 RNA polymerises. A pool of 96 S. typhimurium SL1344 mutants was inoculated into L-Broth (10 ml) containing Kanamycin at 50 μg/ml and grown overnight at 37° C. statically. Chromosomal DNA (20 μg) was prepared from 1.5 ml of culture using the Qiagen DNeasy Kit, and 5 μl (0.5 μg) digested individually with the restriction enzymes HaeIII, HhaI, Hpych4 IV and RsaI (NEB) in their respective NEB buffers in a final volume of 20 μl, overnight at 37° C. The enzymes were then heat denatured at 65° C. for HhaI, Hpych4 IV and RsaI and 80° C. for HaeIII for 20 min. Each 20 μl digest was then self-ligated with T4 DNA ligase (Gibco) in a 100 μl reaction at 4° C. for 48 h. Amplification of the DNA flanking each end of the transposon was achieved by iPCR. iPCR reactions to amplify the SP6 end of the transposon are performed with: oligonucleotide 107 (SEQ ID NO: 4) 5′-CTACCCTGTGGAACACCTACATCT-3′; and one of either oligonucleotide 115 (SEQ ID NO: 5) 5′-ATTACCTCTTTCTCCGCACCCGAC-3′; RsaI or Hpych4IV or oligonucleotide 116 (SEQ ID NO: 6) 5′-CGACATAGATCCGGAACATAATGG-3′; HaeIII or HhaI, depending on the restriction enzyme used (in brackets) to cut the chromosomal DNA. iPCR reactions to amplify the T7 end of the transposon were performed with oligonucleotide 108 (SEQ ID NO: 7) 5′-ACCTACAACAAAGCTCTCATCAACC-3′ and one of either oligonucleotide 117 (SEQ ID NO: 8) 5′-ACAACCTATTAATTTCCCCTCGTC-3′; RsaI, HaeIII or BhaI or oligonucleotide 118 (SEQ ID NO: 9) 5′-ATGTTGGAATTTAATCGCGGCCTC-3′; Hpych4IV, depending on the restriction enzyme used (in brackets) to cut the chromosomal DNA. iPCR reactions were then set up using Qiagen Taq polymerase according to the Qiagen protocol. 4 μl (0.02 μg) of ligation was used as template for each iPCR reaction. The reactions were initially denatured at 94° C. 3′ followed by 30 cycles of 94° C. 30s, 65° C. 90s, 72° C. 90s followed by 7 min extension at 72° C. and then cooling to 4° C. Each of the 8 iPCR's were purified using a Qiagen Gel extraction kit and the DNA eluted in 5 μl water. Each iPCR product was then re-digested with its respective restriction enzyme in a final volume of 50 μl (NEB) overnight 37° C. The digests were then cleaned using a Qiagen Gel extraction kit (following the manufacturers recommended procedure) and the DNA eluted in 50 μl EB pH8.5.

Each digested iPCR product (2 μl) was used as a template for both T7 and SP6 in vitro transcription reactions according to the Ambion protocol. The RNA was cleaned using the Qiagen Rneasy kit and eluted in 50 μl of RNase free water that was then placed in a UV transparent 96 well plate and the absorbance at 260 nm measured.

### Example 6

### Ligation Capture Recovery of Gene Kelly Transposon Ends

A significant advantage of the Gene Kelly (GK) transposon is that it permits the recovery of DNA fragments adjacent to the site of transposon insertion by a method that does not employ PCR, which is ligation capture. Essentially, because of the rarity of the I-SceI and PI-PspI homing endonuclease sites the T7 and SP6 promoter sites that are linked to these sites can be enriched from a pool of DNA by ligation of a biotinylated linker to the cut site followed by purification using Streptavidin linked magnetic beads. A ligation capture experiment was performed on pBAD-GK6A. Plasmid DNA (1 μg) was digested with PI-PspI overnight and the resulting linearised DNA purified using a Qiagen Gel extraction kit. This DNA (400 ng) was then digested overnight with HaeIII and subsequently dephosphorylated using Calf Intestinal Alkaline Phosphatase (Roche). Dephosphorylated DNA was then ligated overnight onto a biotinylated linker, generated by annealing oligonucleotide 113 (SEQ ID NO: 10) 5 ′-biotin-GACGACCTCAGTTACGGTACGATCGGCCACGTAGCTTAT-3′ and oligonucleotide 114 (SEQ ID NO: 11) 5′-phosphate-GCTACGTGGCCGATCGTACCGTAACTGAGGTCGTC-3′.

The ligation was purified using a Qiagen Gel extraction. Biotinylated DNA was then extracted from the ligation using Streptavidin-linked magnetic particles (150 μg; Promega) according to the manufacturers protocol, and the beads finally resuspended in 8 μl of 1×HaeIII restriction buffer containing 10 units of HaeIII (NEB). The digestion was then incubated at 37° C. for 2 hours to remove the T7 RNA promoter from the linker. The beads were removed and an IVT reaction performed on the supernatant using an Ambion T7 Megashortscript kit, according to the manufacturers instructions. IVT products were purified using a Qiagen RNeasy kit, and the products eluted in 50 μl water and read at A₂₆₀.

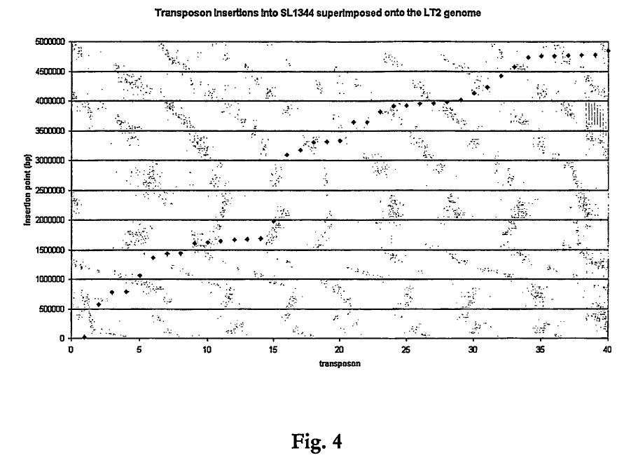

From the sequence data obtained we were able to identity the transposon insertion point in the published Salmonella genome LT2. FIG. 4 shows a graph showing the random distribution of the sites of GK transposon distribution in the LT2 genome for the 46 sequenced mutants.

### Example 7

### Identification of Genes in Salmonela typhimurium Important for Virulence in the Mouse Model of Infection

480 random Gene Kelly transposon mutants were generated in Salmonella typhimurium SL1344. The mutants were grown individually in 1 ml of L-broth in 96-well HTS plates, overnight and subsequently pooled. Chromosomal DNA (78.6 μg) was purified and subsequently restriction digested separately with HaeIII and RsaI and the products purified with Qiagen gel extraction columns. The DNA was divided in half and separate IVT reactions (19.6 μg DNA templates/IVT) were performed with the respective RNA polymerase (SP6 and T7) resulting in the generation of labelled target (SP6 and T7 target was labelled with Cy5 and Cy3, respectively) corresponding to the DNA flanking both ends of transposon insertions.

DNA microarrays were designed based on the entire Salmonella typhimurium LT2 genome sequence, with probes synthesised in both the sense and anti-sense directions. Following hybridisation, the data were extracted from the arrays and analysed to identify those genes disrupted by transposon insertion within the pool of 480 mutants. New microarrays were designed incorporating probes corresponding to each site of transposon insertion.

The mutants were grown individually in 1 ml of L-broth in 96-well HTS plates, overnight. Cultures were pooled (‘Input pool’) and chromosomal DNA purified from 50 ml of the resultant culture (‘Input pool’ DNA). An inoculum of 10⁵ cfu/ml PBS was generated from the ‘input pool’. Mice were inoculated i.v. with 10⁵ cfu of the ‘input pool’ and the infection allowed to proceed for 2.5 days, whereupon the mice were sacrificed and liver and spleen removed. Organs were pulverised in 10 ml water and spread onto 4×120 mm diameter L-Agar plates and incubated overnight at 37° C. Bacteria (‘output pool’) were harvested from these plates by re-suspending the bacterial lawns in 10 ml L-broth/plate. Chromosomal DNA (‘output pool DNA’) was purified from 3 ml of this suspension.

‘Input’ and ‘output pool’ DNA (5 μg) was digested overnight with the restriction endonuclease RsaI, and subsequently cleaned on a Qiagen Gel extraction column. In vitro transcription reactions were set up using the Ambion T7 and SP6 Megascript kits, using 2 μg of digested DNA as template, with Cy3-CTP and Cy5-CTP, respectively, and incubated at 37° C. for 24 hrs. Following the DNaseI step RNA was purified using a Qiagen nucleotide removal kit The RNA from both the SP6 and T7 IVT reactions were then hybridised overnight to a DNA microarray containing probes corresponding to each transposon insertion site. Arrays were washed in 0.6×SSPE X followed by 0.06×SSPE 0.18% PEG200, each wash lasting 5 min. Slides were then dried and analysed using an Agilent microarray scanner. Several mutants in the pool of 96 were analysed using DNA sequencing to ascertain the precise point of transposition within the SL1344 chromosome. One mutant was characterised as containing a transposon within the aroA gene that would lead to the loss of its function. AroA mutants are one of the best genetically defined S. typhimurium vaccine strains (Chatfield et al., 1992; Microb. Pathog 12: 145-151). These mutants are reduced in their ability to survive within susceptible mice compared to its wild type parent strain, such that 3 days post infection bacterial levels are reduced 1000-fold.

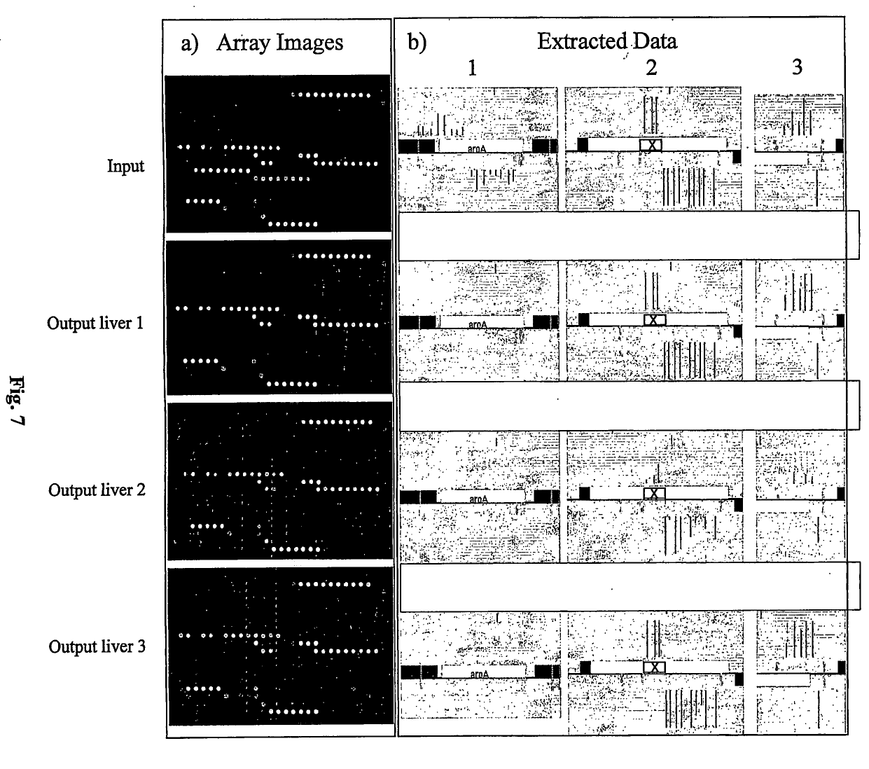

Data from the microarray images were extracted using Agilent's image analysis software and the data fed into a data viewing package. Array data from the input and output hybridisations were compared (see FIG. 7).

Analysis of both the array image and the extracted data from the input pool reveal that target was generated to the aroA gene and that this target hybridised to the expected probes surrounding the site of transposon insertion. Analysis of the corresponding data from all three output pools revealed that significantly less target was hybridised to the probes corresponding to the aroA gene compared to two control mutants with transposons disrupting other loci (gene X and an intergenic region) in the SL1344 genome. These data indicate that the aroA mutant is attenuated in its ability to survive within the mice. Therefore this technique allows the identification of genes that are important for the virulence of S. typhimurium in the mouse model of infection.

### Example 8

### Construction of a Mariner Erm Gene Kelly Transposon

The Gene Kelly R6k ori Kan transposon (i.e. the transposon generated in Example 1) was used as a PCR template with oligonucleotides:

135

(SEQ ID NO: 12)

5′-TAACAGGTTGGCTGATAAGTCCCCGGTCTCCCTATAGTGAG-3′;
and
136

(SEQ ID NO: 13)

5′-TAACAGGTTGGCTGATAAGTCCCCGGTCTCTTCTATAGTGTC-3′.

Insertion sequences are italicised, overlap with the RNA polymerase binding sites present in the Tn5 Gene Kelly construct are in bold. PCR with these oligonucleotides maintain the internal design of Tn5 Gene Kelly transposon comprising the T7 RNA polymerase site adjacent to the homing endonuclease site PI-PspI, and the SP6 RNA polymerase site adjacent to the homing endonuclease site for I-SceI, respectively. PCR was carried out according to protocol for Roche Expand Hi-fidelity Kit.

A first round of PCR, to ensure annealing of the short oligonucleotide overlap sequences with the denatured Tn5 Gene Kelly, was carried out with an initial denaturation step at 94° C. for 3 min, then 5 cycles of 94° C. 30s, 40° C. 90s, 72° C. 2 min 30s, then a further 25 cycles at 94° C. 30s, 55° C. 90s, 72° C. 2 min 30s, final extension of 5 minutes and then cooled to 4° C. The PCR product was separated by gel electrophoresis, and the band (corresponding to the expected product at 2064 bp) cut out and Gel extracted according to the Qiagen gel extraction protocol. The PCR products were ligated (according to Gibco protocol for T4 DNA ligase) into the EcoRV restriction site of pETBlue-1 (Novagen), overnight at 16° C. The ligation mixture was then transformed into E.coli TOP10 cells (Invitrogen). The transformation was then plated out on L-Agar (Sigma) plates containing Kanamycin at 30 μg/ml and incubated overnight at 37° C. 12 colonies were then picked and grown up overnight in LB-Kan 30 μg/ml and 1.5 ml of culture was used for DNA-mini-preps according to the Qiagen protocol. The DNA sequence of the resulting constructs was then determined on a Beckman CEQ DNA sequencer, (using manufacturers recommended conditions) with the supplied pETBlue-1 Down and Up sequencing primers. The sequence of clone 5 pETBlueHGK5) was found to contain the expected DNA sequence.

### Example 9

### Cloning of an Erythromycin Resistance Marker into Mariner Erm Gene Kelly

A plasmid containing an erythromycin resistance marker, originally from plasmid pIL253 (Simon & Chopin (1988), Biochimie 70 (4), 559-566), was cloned into pETBlueHGK5 to provide a selection marker for the Transposon following integration into the chromosome of selected Gram-positive bacteria The erythromycin gene was amplified from pIL253 using primers:
5′ erm 5′-GATATCGAAGCAAACTTAAGAGTGT-3′ (SEQ ID NO: 14)
3′ erm 5′-GATATCTACAAAAGCGACTCATAGA-3′ (SEQ ID NO: 15)
   and cloned into pCR2.1 Invitrogen). The EcoRV restriction sites (underlined) were used to remove the erythromycin cassette from this vector following digestion with the respective restriction enzyme (NEB). The resistance marker (871 bp) was then purified by agarose gel electrophoresis followed by the Qiagen gel extraction protocol. The plasmid pETBlueHGK5 was linearised (5540 bp) with the restriction enzyme HincII (NEB), and dephosphorylated using Shrimp Alkaline phosphatase (Roche) and then purified by agarose gel electrophoresis followed by the Qiagen gel extraction protocol. The products were ligated (according to Gibco protocol for T4 DNA ligase) overnight at 16° C. The ligation mixture was then transformed into E.coli TOP10 cells (Invitrogen). The transformation was then plated out on L-Agar (Sigma) plates containing Kanamycin at 30 μg/ml and erythromycin at 200 μg/ml and incubated overnight at 37° C. 6 colonies were then picked and grown up overnight in LB-Kan 30 μg/ml and 1.5 ml of culture was used for DNA-mini-preps according to the Qiagen protocol. The DNA sequence of the resulting constructs was then determined on a Beckman CEQ DNA sequencer, (using manufacturers recommended conditions) with the sequencing primers:
12 5′-AAG ATA CTG CAC TAT CAA CAC ACT C-3′ (SEQ ID NO: 16)
13 5′-ATT AAG AAG GAG TGA TTA CAT GAA C-3′ (SEQ ID NO: 17)
   as well as the pETBlue-1 Down and Up sequencing primers (Novagen).

The sequence of clone 3 (pHGK5erm3) was found to contain the desired DNA sequence. The transposon contained within this plasmid was named the Mariner Erm Gene Kelly transposon.

### Example 10

### Mutagenesis of Staphylococcus aureus with the Mariner Erm Gene Kelly

A protocol (see below; Generation of a Mariner Erm Gene Kelly transposon library) and strains for mutagenesis of Staphylococcus aureus was obtained from Prof. S. Foster (Sheffield University, MTA). The protocol results in Mariner Transposon integration into the S. aureus chromosome, following introduction of two temperature sensitive plasmids into the recipient strain (one bearing the Mariner Transposon; TS1, the other the Mariner Transposase gene; pSPT246), and induction of transposition.

### Example 11

### Construction of S. aureus Strains Replicating Mariner Erm Gene Kelly

Plasmid TS1 was digested with the restriction endonuclease BamHI (NEB), dephosphorylated with alkaline phosphatase (Roche) and purified by agarose gel electrophoresis followed by the Qiagen gel extraction protocol. The Mariner Erm Gene Kelly was removed from pHGK5erm3 by digestion with the restriction endonucleases BglII and SmaI (NEB), and purified by agarose gel electrophoresis followed by the Qiagen gel extraction protocol. The products were ligated (according to Gibco protocol for T4 DNA ligase) overnight at 16° C. The ligation mixture was then transformed into E.coli PIR1 cells (Epicentre). The transformed cells were then plated on L-Agar (Sigma) plates containing Kanamycin at 30 μg/ml and incubated overnight at 37° C. 12 colonies were then picked and grown up overnight in LB-Kan 30 μg/ml and 1.5 ml of culture was used for DNA-mini-preps according to the Qiagen protocol. Restriction digestion with EcoRI and XbaI (NEB), followed by agarose gel electrophoresis indicated that one clone contained the correct plasmid (pMARGK2b). DNA sequencing of pMARGK2b was performed on a Beckman CEQ DNA sequencer, (using manufacturers recommended conditions) with the sequencing primers:
12 5′-AAG ATA CTG CAC TAT CAA CAC ACT C-3′ (SEQ ID NO: 16)
107 5′-CTACCCTGTGGAACACCTACATCT-3′ (SEQ ID NO: 4),
   and found to contain the desired DNA sequence. This plasmid also contains a temperature sensitive origin for replication in S. aureus at temperatures of 30° C. or below, plus a chloramphenicol resistance marker providing resistance in S. aureus at 5 μg/ml.

pMARGK2b was introduced into S. aureus RN4220 by electroporation (0.5 μg pMARGK2b: 2.3 kV (0.1 cm cuvette), 25 μF, 100Ω) using a Gene Pulser (Biorad). The electroporation was plated out onto BHI-agar (Oxoid) containing chloramphenicol and erythromycin at 5 μg/ml. 6 colonies were then picked and grown up overnight at 30° C. in BHI containing erythromycin and chloramphenicol at 5 μg/ml and 1.5 ml of culture was used for plasmid DNA-mini-preps according to the Qiagen protocol. Plasmid DNA was restriction digested with EcoRI and XbaI and analysed by agarose gel electrophoresis. The restriction pattern of all 6 clones matched that obtained following the same digestion of pMARGK2b isolated from E. coli.

Transducing bacteriophage φ11 was propagated (Novick, R. P. 1991. Genetic systems in staphylococci. Methods Enzymol. 204:587-636) from S. aureus RN4220 pMARGK1-6 and used to transduce the 6 plasmids into virulent S. aureus SH1000, generating S. aureus SH1000 pMARGK1-6. 2 colonies were then picked from each transduction and grown up overnight at 30° C. in BHI containing erythromycin and chloramphenicol at 5 μg/ml and 1.5 ml of culture was used for plasmid DNA-mini-preps according to the Qiagen protocol. Plasmid DNA was restriction digested with EcoRI and XbaI and analysed by agarose gel electrophoresis. The restriction pattern of clones 1-5 matched that obtained following the same digestion of pMARGK2b isolated from E. coli. S. aureus SH1000 pMARGK3a was chosen as the parent strain for the generation of a transposon library.

### Example 12

### Generation of S. aureus Strain SH1000 Containing Mariner Erm Gene Kelly, and the Mariner Transposase Gene

Plasmid pSPT246, containing the Mariner transposase and a tetracycline resistance gene, was introduced into S. aureus SH1000 pMARGK3a by transducing the plasmid from S. aureus SH1000 pSPI246 (isolate 3a) using φ11-transducing bacteriophage. Following transduction, bacteria were propagated on agar containing chloramphenicol, erythromycin and tetracycline all at 5 μg/ml at 30° C. for 48 h. Transduction yielded approximately 2000 colonies. All colonies were extracted from the top agar and inoculated into 600 ml BHI broth containing chloramphenicol, erythromycin and tetracycline all at 5 μg/ml and incubated o/n at 30° C. Bacteria (100 ml) were centrifuged at 4000×g for 5 min and resuspended in 5 ml BHI broth containing 50% (v/v) glycerol and stored in 0.5 ml aliquots at −80° C.

### Example 13

### Generation of a Mariner Erm Gene Kelly Transposon Library in S. aureus SH1000

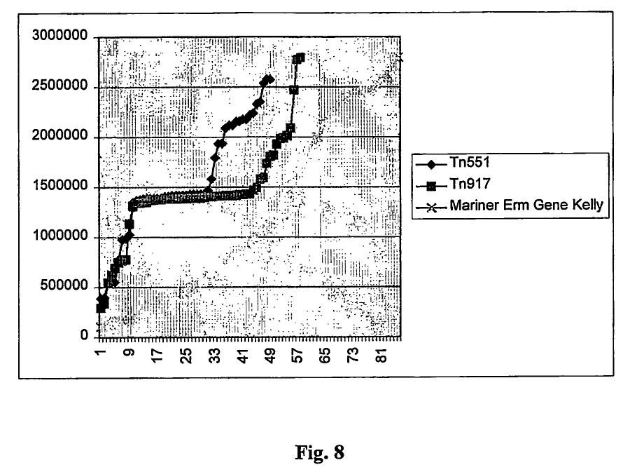

S. aureus SH1000 pMARGK3a pSRT146-3a (0.5 ml glycerol stock from above) was inoculated into 100 ml of room temperature BHI broth containing chloramphenicol, erythromycin and tetracyclin all at 5 μg/ml and incubated at 37° C. until the culture reached an A₆₀₀ of 0.4. 30 ml of this culture was centrifuged at 4000×g for 5 min and the pellet resuspended in 600 ml BHI broth containing 5 μg/ml erythromycin at 44° C. This culture was incubated at 44° C. until the culture reached an A₆₀₀ of 0.4. 30 ml of this culture was centrifuged at 4000×g for 5 min and the pellet resuspended in 600 ml BHI broth containing 5 μg/ml erythromycin at 44° C. This culture was incubated at 44° C. overnight. The resulting bacteria were tested and confirmed as sensitive to tetracyclin and chloramphenicol whilst maintaining resistance to erythromycin. This indicated that transposition of Mariner Erm Gene Kelly into the S. aureus SH1000 chromosome had occurred. Chromosomal DNA was prepared from 200 ml of this culture for subsequent TMDH protocols. Glycerol stocks of this culture were prepared by centrifugation of 100 ml of this culture at 4000×g for 5 min and resuspending the bacterial pellet in BHI containing 50% glycerol. Chromosomal DNA was prepared from 112 colonies generated from the library generation protocol and sequenced using primer 199 5′ TAGCCAGTTTCGTCGTTAAATGCCC 3′ (SEQ ID NO: 18) that binds 310 bases 5′ of the end of the transposon. Interpretable DNA sequence from 86 strains where the DNA flanking the end of the transposon matched S. aureus DNA sequences located in the public databases, indicating transposition of the Mariner Erm Gene Kelly transposon into the S. aureus SH1000 chromosome. A comparison of the location of these transposon insertions relative to the complete chromosomal DNA sequence of S. aureus strain MW2 is shown in FIG. 8. This reveals the random nature of Mariner transposition into the S. aureus chromosome. This is compared to 59 S. aureus SH1000 Tn917 mutants, and 50 S. aureus SH1000 Tn551 mutants made using the transposons Tn917 and Tn551, respectively. Analysis of this data reveals that both Tn551 and Tn917 have a transposition hotspot encompassing a region of approximately 60 kb where about half of the mutants derived from each transposon were located. Mutants generated using Mariner Erm Gene Kelly do not appear to have such a sequence preference and therefore libraries generated using this system will be of a much higher quality than those generated using either Tn917 or Tn551. In essence Mariner is much more suited to the generation of libraries suited to TMDH analysis of the S. aureus genome.

### Example 14

### Evaluation of the Mariner Erm Gene Kelly for TMDH

(i) T7 and SP6 RNA Polymerase Evaluation

In order to evaluate the novel transposon as suitable for the TMDH protocol, iPCR reactions amplifying both ends of the transposon as well as the DNA flanking the site of transposition from one of the sequenced transposon mutants were used as template in an in vitro transcription (IVT) reactions using both SP6 (SP6 Megascript kit; Ambion) and T7 (T7 Megashortscipt kit, Ambion) RNA polymerases, according to Ambion protocols. RNA produced was purified using the Qiagen RNeasy mini Kit, and the amount of RNA produced measured at A₂₆₀. Transcription was observed both with the SP6 and T7 IVT reactions, but only when the specific RNA polymerase was included in a reaction containing the iPCR product bearing the cognate RNA polymerase binding site. This indicates no transcription occurs as a consequence of exposure of the T7 and SP6 RNA polymerases, respectively, to the SP6 and T7 RNA polymerase promoter sequences.

1-47. (canceled)

48. A transposon which comprises an RNA polymerase recognition site and a homing endonuclease recognition site.

49. A transposon according to claim 48 which comprises two RNA polymerase recognition sites.

50. A transposon according to claim 49, wherein the two RNA polymerase recognition sites are diverse.

51. A transposon according to claim 50, wherein the two diverse RNA polymerase recognition sites are two of a T7 RNA polymerase recognition site, an SP6 RNA polymerase recognition site or a T3 RNA polymerase recognition site.

52. A transposon according to claim 48 which comprises two homing endonuclease recognition sites.

53. A transposon according to claim 52, wherein the two homing endonuclease recognition sites are diverse.

54. A transposon according to claim 53, wherein the two diverse homing endonuclease recognition sites are an I-SceI recognition site and a PI-PspI recognition site.

55. A transposon according to claim 48 which further comprises a bacterial origin of replication.

56. A transposon according to claim 48 which is a modified Tn5 transposon or a modified Mariner transposon.

57. A method for identifying an essential gene of an organism, which method comprises:
(i) providing a library of transposon insertion mutants of the said organism, wherein the transposon is a transposon according to claim 48;
(ii) isolating chromosomal DNA from the library of (i);
(iii) digesting the chromosomal DNA with a restriction endonuclease that is capable of cutting 5′ to the RNA polymerase recognition site(s) in the transposon and 3′ to the RNA polymerase recognition site(s) in the chromosomal DNA flanking the transposon insertion site;
(iv) transcribing the resulting digested DNA from the RNA polymerase recognition site(s) in the said DNA;
(v) hybridizing the resulting RNA with an oligonucleotide array; and
(vi) identifying at least one probe on the oligonucleotide array which corresponds to an essential gene of the organism.

58. A method according to claim 57, wherein a labeled ribonucleotide is present when transcribing the digested DNA in step (iv).

59. A method according to claim 58, wherein step (v) is replaced by:
(v)′ reverse transcribing the resulting RNA; and
(v)″ hybridizing the resulting cDNA with an oligonucleotide array.

60. A method according to claim 59, wherein a labeled deoxyribonucleotide is present when reverse transcribing the RNA in step (v)′.

61. A method according to claim 57, wherein:
(a) the transposon comprises two RNA polymerase recognition sites which are diverse;
(b) step (iv) is carried out by transcribing one aliquot of the digested DNA with a first RNA polymerase and transcribing a second aliquot of the digested DNA with a second different RNA polymerase; and
(c) step (v) is carried out by hybridizing the two resulting RNA pools with the same oligonucleotide array or separately with two copies of the same oligonucleotide array.

62. A method according to claim 61, wherein in step (b) the two aliquots of digested DNA are each transcribed in the presence of a different labeled ribonucleotide.

63. A method according to claim 59, wherein:
(a) the transposon comprises two RNA polymerase recognition sites which are diverse;
(b) step (iv) is carried out by transcribing one aliquot of the digested DNA with a first RNA polymerase and transcribing a second aliquot of the digested DNA with a second different RNA polymerase; and
(c) step (v)″ is carried out by hybridizing the two resulting cDNA pools with the same oligonucleotide array or separately with two copies of the same oligonucleotide array.

64. A method according to claim 63, wherein the two aliquots of RNA resulting from step (b) are each reverse transcribed using a different labeled deoxyribonucleotide.

65. A method according to claim 57, wherein:
(a) aliquots of the chromosomal DNA are digested separately with different restriction endonucleases in step (iii);
(b) each of the restriction endonucleases is capable of cutting 5′ to the RNA polymerase recognition site(s) in the transposon and 3′ to the RNA polymerase recognition site(s) in the chromosomal DNA flanking the transposon insertion site; and
(c) each aliquot is subsequently treated separately in steps (iv) to (vi).

66. A method according to claim 65, wherein two or three aliquots of the chromosomal DNA are each digested separately with different restriction endonucleases.

67. A method according to claim 57, wherein step (iii) is replaced by:
(iii)′ digesting the chromosomal DNA with a homing endonuclease which is capable of cutting 5′ to RNA polymerase recognition site(s) in the transposon;
(iii)″ digesting the chromosomal DNA with a restriction endonuclease that is capable of cutting 3′ to the RNA polymerase recognition site(s) in the chromosomal DNA flanking the transposon insertion site; and
(iii)′″ ligating the digested DNA with a biotinylated linker; and
(iii)″″ recovering the digested DNA using streptavidin-coated particles.

68. A method for identifying a conditional essential gene of an organism, which method comprises:
(a) providing a first sample of a library of transposon insertion mutants of the said organism (input library);
(b) providing a second sample of the library and subjecting that sample to a conditional restraint;
(c) collecting the mutants that survive the conditional restraint in step (ii) to give a second library (output library);
(d) carrying out a method according to steps (ii) to (iv) of claim 57 on the input library from step (a) and on the output library from step (c);
(e) hybridizing the transcribed RNA derived from the input library and from the output library separately to copies of the same oligonucleotide array or, if the RNA derived from the two libraries is differentially labeled, to the same oligonucleotide array; and
(f) identifying at least one probe on the oligonucleotide array(s) which corresponds to a conditional essential gene of the organism.

69. A method for identifying a conditional essential gene of an organism, which method comprises:
(a) providing a first sample of a library of transposon insertion mutants of the said organism (input library);
(b) providing a second sample of the library and subjecting that sample to a conditional restraint;
(c) collecting the mutants that survive the conditional restraint in step (ii) to give a second library (output library);
(d) carrying out a method according to steps (ii) to (v)′ of claim 59 on the input library from step (a) and on the output library from step (c);
(e) hybridizing the reverse transcribed cDNA derived from the input library and from the output library separately to copies of the same oligonucleotide array or, if the cDNA derived from the two libraries is differentially labeled, to the same oligonucleotide array; and
(f) identifying at least one probe on the oligonucleotide array(s) which corresponds to a conditional essential gene of the organism.

70. A method according to claim 68, wherein the organism is a bacterium and the conditional restraint is growth of that bacterium in its host.

71. A method according to claim 57 or 68, wherein the oligonucleotide array comprises probes which are from 9 to 150 bp in length and/or comprises 1 probe for every 60 to 250 bp of the locus or loci represented on the array.

72. A method for identifying an inhibitor of transcription and/or translation of an essential or conditional essential gene and/or an inhibitor of activity of a polypeptide encoded by a said gene, which method comprises:
(a) identifying an essential or conditional essential gene by a method according to claim 57 or 68; and
(b) determining whether a test substance can inhibit transcription and/or translation of a gene identified in step (a) and/or activity of a polypeptide encoded by a said identified gene, thereby to identify a said inhibitor.

73. An inhibitor identified by a method according to claim 72.

74. An inhibitor according to claim 73 which is an antibody.

75. An inhibitor according to claim 74 which is a monoclonal antibody.

76. A pharmaceutical composition comprising an inhibitor according to claim 73 wherein the essential or conditional essential gene is a bacterial, fungal or eukaryotic parasite essential or conditional essential gene and a pharmaceutically acceptable carrier or diluent.

77. A method of treating a host suffering from a bacterial, fungal or eukaryotic parasite infection, which method comprises the step of administering to the host a therapeutically effective amount of an inhibitor according to claim 73 wherein the essential or conditional essential gene is a bacterial, fungal or eukaryotic parasite essential or conditional essential gene.

78. A method for the preparation of a pharmaceutical composition, which method comprises:
(a) identifying an inhibitor of transcription and/or translation of an essential or conditional essential gene of an organism and/or an inhibitor of activity of a polypeptide encoded by a said gene by a method according to claim 72 wherein the essential or conditional essential gene is a bacterial, fungal or eukaryotic parasite essential or conditional essential gene; and
(b) formulating the inhibitor thus identified with a pharmaceutically acceptable carrier or diluent.

79. A method for treating a host suffering from a bacterial, fungal or eukaryotic parasite infection, which method comprises:
(a) identifying an inhibitor of transcription and/or translation of an essential or conditional essential gene of an organism and/or an inhibitor of activity of a polypeptide encoded by a said gene by a method according to claim 72 wherein the essential or conditional essential gene is a bacterial, fungal or eukaryotic parasite essential or conditional essential gene;
(b) formulating the inhibitor thus identified with a pharmaceutically acceptable carrier or diluent; and
(c) administering to the host a therapeutically effective amount of an inhibitor thus formulated.

80. An inhibitor according to claim 73, wherein the essential or conditional essential gene is a plant bacterial, plant fungal, plant pest or plant essential or conditional essential gene.

81. A bacterium attenuated by a non-reverting mutation in one or more genes identified by a method according to claim 68.

82. A vaccine comprising a bacterium according to claim 81 and a pharmaceutically acceptable carrier or diluent.

83. A vaccine comprising a peptide encoded by an essential or conditional essential gene sequence identified by a method according to claim 57 or 68.

84. A method for raising an immune response in a mammalian host, which method comprises the step of administering to the host a bacterium according to claim 81 or a peptide encoded by an essential or conditional essential gene sequence identified by a method according to claim 57 or 68.

85. A method for preparing an attenuated bacterium, which method comprises:
(a) identifying a conditional essential gene in a bacterium by a method according to claim 68; and
(b) introducing a non-reverting mutation into a thus-identified conditional essential gene of the bacterium, thereby to attenuate the bacterium.

86. A method for the preparation of a vaccine, which method comprises:
(a) identifying a conditional essential gene in a bacterium by a method according to claim 68;
(b) introducing a non-reverting mutation into a thus-identified conditional essential gene of the bacterium, thereby to attenuate the bacterium; and
(c) formulating the attenuated bacterium with a pharmaceutically acceptable carrier or diluent.

87. A method for the preparation of a vaccine, which method comprises:
(a) identifying an essential or conditional essential gene sequence by a method according to claim 57 or 68; and
(b) formulating a peptide encoded by a thus-identified essential or conditional essential gene sequence with a pharmaceutically acceptable carrier or diluent.

88. A method for raising an immune response in a mammalian host, which method comprises:
(a) identifying a conditional essential gene in a bacterium by a method according to claim 68;
(b) introducing a non-reverting mutation into a thus-identified conditional essential gene of the bacterium, thereby to attenuate the bacterium;
(c) formulating the attenuated bacterium with a pharmaceutically acceptable carrier or diluent; and administering to the host the attenuated bacterium thus formulated.

89. A method for raising an immune response in a mammalian host, which method comprises:
(a) identifying an essential or conditional essential gene sequence by a method according to claim 57 or 68;
(b) formulating a peptide encoded by a thus-identified essential or conditional essential gene sequence with a pharmaceutically acceptable carrier or diluent; and
(c) administering to the host the peptide thus formulated.


SEQUENCE LISTING (18 sequences)

SEQ ID NO: 1 - DNA, 2044 bp, Artificial Sequence
  Transposon
ctgtctcttatacacatctcttctatagtgtcacctaaatagggataacagggtaatgaattcgttaatacagatgtaggtgttccacagggtagccagcagcatcctgcgatgcagatccggatgccatttcattacctctttctccgcacccgacatagatccgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatctgccatttcattacctctttctccgcacccgacatagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagggtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggcggggatcctctagagtcgactggcaaacagctattatgggtattatgggtaatacgactcactatagggagatgtgtataagagacag

SEQ ID NO: 2 - DNA, 85 bp, Artificial Sequence
  Oligonucleotide
cagctgtctcttatacacatctccctatagtgagtcgtattacccataatacccataatagctgtttgccagtcgactctagagg

SEQ ID NO: 3 - DNA, 80 bp, Artificial Sequence
  Oligonucleotide
cagctgtctcttatacacatctcttctatagtgtcacctaaatagggataacagggtaatgaattcgttaatacagatgt

SEQ ID NO: 4 - DNA, 24 bp, Artificial Sequence
  Oligonucleotide
ctaccctgtggaacacctacatct

SEQ ID NO: 5 - DNA, 24 bp, Artificial Sequence
  Oligonucleotide
attacctctttctccgcacccgac

SEQ ID NO: 6 - DNA, 24 bp, Artificial Sequence
  Oligonucleotide
cgacatagatccggaacataatgg

SEQ ID NO: 7 - DNA, 25 bp, Artificial Sequence
  Oligonucleotide
acctacaacaaagctctcatcaacc

SEQ ID NO: 8 - DNA, 24 bp, Artificial Sequence
  Oligonucleotide
acaacctattaatttcccctcgtc

SEQ ID NO: 9 - DNA, 24 bp, Artificial Sequence
  Oligonucleotide
atgttggaatttaatcgcggcctc

SEQ ID NO: 10 - DNA, 39 bp, Artificial Sequence
  Oligonucleotide
gacgacctcagttacggtacgatcggccacgtagcttat

SEQ ID NO: 11 - DNA, 35 bp, Artificial Sequence
  Oligonucleotide
gctacgtggccgatcgtaccgtaactgaggtcgtc

SEQ ID NO: 12 - DNA, 41 bp, Artificial Sequence
  Oligonucleotide
taacaggttggctgataagtccccggtctccctatagtgag

SEQ ID NO: 13 - DNA, 42 bp, Artificial Sequence
  Oligonucleotide
taacaggttggctgataagtccccggtctcttctatagtgtc

SEQ ID NO: 14 - DNA, 25 bp, Artificial Sequence
  Oligonucleotide
gatatcgaagcaaacttaagagtgt

SEQ ID NO: 15 - DNA, 25 bp, Artificial Sequence
  Oligonucleotide
gatatctacaaaagcgactcataga

SEQ ID NO: 16 - DNA, 25 bp, Artificial Sequence
  Oligonucleotide
aagatactgcactatcaacacactc

SEQ ID NO: 17 - DNA, 25 bp, Artificial Sequence
  Oligonucleotide
attaagaaggagtgattacatgaac

SEQ ID NO: 18 - DNA, 25 bp, Artificial Sequence
  Oligonucleotide
tagccagtttcgtcgttaaatgccc